(19) United States
(12) Patent Application Publication
(10) Pub. No.: US 2015/0044251 A1
(43) Pub. Date: Feb. 12, 2015

(54) STABLE COMPOSITIONS FOR IMMUNISING AGAINST STAPHYLOCOCCUS AUREUS

(71) Applicant: NOVARTIS AG, Basel (CH)

(72) Inventors: Mario Contorni, Siena (IT); Lorenzo Tarli, Siena (IT); Anna Coslovi, Castellina in Chianti (IT); Michele Sotgiu, Colle Val D'Elsa (IT)

(21) Appl. No.: 14/366,362

(22) PCT Filed: Dec. 21, 2012
(86) PCT No.: PCT/EP2012/076609

§ 371 Date: Jun. 18, 2014

Related U.S. Application Data
(60) Provisional application No. 61/580,191, filed on Dec. 23, 2011.

(51) Int. Cl. A61K 39/085

(52) U.S. Cl. CPC A61K 39/085
USPC 424/192.1; 424/243.1

(57) ABSTRACT

Adding stabilizing additives to immunogenic compositions is effective in enhancing antigen stability. Suitable stabilizing additives include EDTA (ethylenediaminetetraacetic acid), sucrose, arginine, protease inhibitors, glycerol and/or citrate.

This application claims the benefit of U.S. provisional application 61/580.191 filed Dec. 23, 2011, the complete contents of all of which are hereby incorporated herein by reference for all purposes

### TECHNICAL FIELD

This invention relates to immunogenic compositions comprising antigens derived from Staphylococcus aureus and to their use in immunisation.

### BACKGROUND ART

S.aureus is a Gram-positive spherical bacterium and is the leading cause of bloodstream, lower respiratory tract, skin and soft tissue infections. It causes a range of illnesses from minor skin infections to life-threatening diseases, and annual US of mortality associated with S.aureus exceeds that of any other infectious disease, including HIV/AIDS.

There is currently no authorised vaccine against S.aureus. A vaccine based on a mixture of surface polysaccharides from bacterial types 5 and 8, StaphVAX™, failed to reduce infections when compared to the placebo group in a phase III clinical trial in 2005.Reference 1 reports data on the “V710” vaccine from Merck and Intercell which is based on a single antigen, IsdB, a conserved iron-sequestering cell-surface protein [2,3].

Reference 4 discloses various S.aureus antigens and their combinations, including “Combo-1” (a mixture of EsxA, EsxB, a mutant Hla, Sta006, and Sta011) and “Combo-2” (a mixture of EsxA, EsxB, IsdA, Sta006, and Sta011). In further work on these antigens the present inventors have observed that S.aureus polypeptide antigens can be unstable in a simple buffer solution. Instability of the antigens is undesirable because (1) it does not allow vaccines to be stored for a long period of time before administration (2) the degradation products may be harmful (e g. inhibitory) when administered, and (3) inconsistency of vaccines from batch to batch does not meet quality and regulatory approval requirements. Therefore it is an object of the invention to stabilize S.aureus polypeptide antigens in immunogenic compositions.

### DISCLOSURE OF THE INVENTION

The inventors have found that adding stabilizing additives to vaccine formulations is effective in enhancing antigen stability. One suitable stabilizing additive is EDTA, as this was shown to be particularly effective in stabilizing the antigens. The inventors believe that EDTA might play a role in inhibiting redox reactions in particular by chelating metal ions involved in the mechanisms of degradation of the antigens or by inhibiting metalloproteases from degrading the antigens. The inventors have also shown that the presence of low concentrations of EDTA (e.g. below 100 mM) in vaccine formulations does not have a significant impact on the thermal characteristic of the vaccine and does not introduce any undesired plasticizing effect, thus meaning that EDTA-containing compositions can be lyophilized to further enhance storage stability.

Thus the invention provides an immunogenic composition comprising an EsxA antigen, an EsxB antigen and a stabilizing additive. The composition can be in aqueous form, in which case it ideally has a pH of between 5 and 6.5.The composition may also include an adjuvant e.g. an aluminium salt.

The combination of using EDTA and a pH between 5 and 6.5 provides a synergistic effect in stabilizing the antigens of a S.aureus vaccine. Preferably, the pH is about 6.

The EsxA and EsxB antigens can be combined as a hybrid polypeptide, as discussed below, e.g. an EsxAB hybrid with an EsxB antigen downstream of EsxA antigen. The EsxAB hybrid polypeptide can exist in a monomeric or an oligomeric form. The oligomer can be a dimer, trimer, or more.

The invention also provides an EsxAB hybrid polypeptide in a dimeric form. Under some conditions the monomeric and dimeric forms of EsxAB are in equilibrium in solution. The dimeric form is formed by the dimerization of two monomeric molecules through the sulphide group of the unique cysteine residue of each EsxAB monomer. The monomeric form is preferred in immunogenic compositions because it is more stable than the dimeric form, and production of the dimeric form (e.g. by oxidation reactions) is inconsistent and so leads to purity variations. The inventors have observed that EDTA stabilizes the EsxAB monomeric form and keeps a high total selectivity of the formulation (i.e. a high proportion of monomeric EsxAB relative to total EsxAB).

The immunogenic composition of the invention may further comprise additional S.aureus antigens, in particular, Sta006, Sta011, and Hla. These antigens are discussed in detail in reference 4. A particularly useful composition of the invention includes all five of these antigens (i.e. EsxA, EsxB, Sta006, Sta011 and Hla, preferably with a non-toxic mutant form of Hla).A simple combination of Sta006, Sta011, and Hla with EsxABis not fully stable in aqueous conditions in the absence of a stabilizing additive. The inventors believe that EsxAB, together with Sta006 and Sta011, perform redox reactions in the buffer solution. Addition of EDTA and adjusting the pH of the composition to around pH 6 maintainsEsxAB in its monomeric form, and minimizes interference with the other components.

In some embodiments of the invention, the further antigens can be polypeptides and/or saccharides. For example, they can usefully also include one or more S.aureus capsular saccharide conjugate(s) e.g. against a serotype 5 and/or a serotype 8 strain. In other embodiments, the composition includes no additional staphylococcal polypeptide antigens. In other embodiments, the composition includes no additional staphylococcal antigens. In yet another embodiment, the composition includes no additional antigens.

The invention also provides a lyophilizate of the immunogenic composition of the invention. This lyophilizate can be reconstituted with aqueous material to provide an aqueous immunogenic composition of the invention. For administration, the lyophilizate is thus reconstituted with a suitable liquid diluent (e.g. a buffer, saline solution, wfi). The liquid diluent can include an adjuvant e.g. an aluminium salt or an oil-in-water emulsion adjuvant.

The invention also provides alyophilizate which comprises EDTA and at least one antigen. The antigen(s) is/are preferably polypeptide(s).

The invention also provides an oligomer of a Sta006 antigen, and also immunogenic compositions comprising such oligomers. The oligomer can be a dimer, trimer, tetramer, or higher. An oligomer may comprise a Ca⁺⁺ ion, and a composition comprising Sta006 oligomers may comprise 5-500 mMCa⁺⁺ ions.

The invention also provides a heterodimer of a Sta006 antigen and a Sta011 antigen. This dimer may comprise a Ca⁺⁺ ion, and a composition comprising such dimers may comprise 5-500 mMCa⁺⁺ ions.

S.aureus Antigens

EsxA

The ‘EsxA’ antigen is annotated as ‘protein’. In the NCTC 8325 strain EsxA is SAOUHSC_{—}00257 and has amino acid sequence SEQ ID NO: 10 (GI:88194063).

EsxA antigens of the invention can elicit an antibody (e.g. when administered to a human) that recognises SEQ ID NO: 10 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 10; and/or (b) comprising a fragment of at least ‘n’ consecutive amino acids of SEQ ID NO: 10, wherein ‘n’ is 7 or more (e.g. 8, 10, 12, 14, 16, 18, 20, 25, 30, 35, 40, 50, 60, 70, 80, 90 or more). These EsxA proteins include variants of SEQ ID NO: 10. Preferred fragments of (b) comprise an epitope from SEQ ID NO: 10. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 10 while retaining at least one epitope of SEQ ID NO: 10. Other fragments omit one or more protein domains.

EsxB

The ‘EsxB’ antigen is annotated as ‘EsxB’. In the NCTC 8325 strain EsxB is SAOUHSC_{—}00265 and has amino acid sequence SEQ ID NO: 11 (GI:88194070).

EsxB antigens of the invention can elicit an antibody (e.g. when administered to a human) that recognises SEQ ID NO: 11 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 11; and/or (b) comprising a fragment of at least ‘n’ consecutive amino acids of SEQ ID NO: 11, wherein ‘n’ is 7 or more (e.g. 8, 10, 12, 14, 16, 18, 20, 25, 30, 35, 40, 50, 60, 70, 80, 90, 100or more). These EsxB proteins include variants of SEQ ID NO: 11. Preferred fragments of (b) comprise an epitope from SEQ ID NO: 11. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 11 while retaining at least one epitope of SEQ ID NO: 11. Other fragments omit one or more protein domains.

EsxAB

Where a composition includes both EsxA and EsxB antigens, these may be present as a single polypeptide (i.e. as a fusion polypeptide). Thus a single polypeptide can elicit antibodies (e.g. when administered to a human) that recognise both SEQ ID NO: 10 and SEQ ID NO: 11. The single polypeptide can include: (i) a first polypeptide sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 10 and/or comprising a fragment of at least ‘n’ consecutive amino acids of SEQ ID NO: 10, as defined above for EsxA; and (ii) a second polypeptide sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 11 and/or comprising a fragment of at least ‘n’ consecutive amino acids of SEQ ID NO: 11, as defined above for EsxB. The first and second polypeptide sequences can be in either order, N- to C-terminus. SEQ ID NOs: 151 (‘EsxAB’) and 152 (‘EsxBA’) are examples of such polypeptides, both having hexapeptide linkers ASGGGS (SEQ ID NO: 173). Another ‘EsxAB’ hybrid comprises SEQ ID NO: 241, which may be provided with a N-terminus methionine (e.g. SEQ ID NO: 250).

Thus a useful polypeptide comprises an amino acid sequence (a) having 80% or more identity (e.g. 80%, 85%, 90%, 91%, 92%, 93%, 94%, 95%, 96%, 97%, 98%, 99%, 99.5% or more) to SEQ ID NO: 241; and/or (b) comprising both a fragment of at least ‘n’ consecutive amino acids from amino acids 1-96 of SEQ ID NO: 241 and a fragment of at least ‘n’ consecutive amino acids from amino acids 103-205 of SEQ ID NO: 241, wherein ‘n’ is 7 or more (e.g. 8, 10, 12, 14, 16, 18, 20, 25, 30, 35, 40, 50, 60, 70, 80, 90, 100, 150, 200, 250 or more). These polypeptides (e.g. SEQ ID NO: 250) can elicit antibodies (e.g. when administered to a human) which recognise both the wild-type staphylococcal protein comprising SEQ ID NO: 10 and the wild-type staphylococcal protein comprising SEQ ID NO: 11. Thus the immune response will recognise both of antigens EsxA and EsxB. Preferred fragments of (b) provide an epitope from SEQ ID NO: 10 and an epitope from SEQ ID NO: 11.

Sta006

The ‘Sta006’ antigen is annotated as ‘ferrichrome-binding protein’, and has also been referred to as ThuD2′ in the literature [5]. In the NCTC 8325 strain Sta006 is SAOUHSC_{—}02554 and has amino acid sequence SEQ ID NO: 42 (GI:88196199). In the Newman strain it is nwmn_{—}2185 (GI:151222397).Sta006 used with the invention can elicit an antibody (e.g. when administered to a human) that recognises SEQ ID NO: 42 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 42; and/or (b) comprising a fragment of at least ‘n’ consecutive amino acids of SEQ ID NO: 42, wherein ‘n’ is 7 or more (e.g. 8, 10, 12, 14, 16, 18, 20, 25, 30, 35, 40, 50, 60, 70, 80, 90, 100, 150, 200, 250 or more). These Sta006 proteins include variants of SEQ ID NO: 42. Preferred fragments of (b) comprise an epitope from SEQ ID NO: 42. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 42 while retaining at least one epitope of SEQ ID NO: 42. The first 17 N-terminal amino acids of SEQ ID NO: 42 can usefully be omitted (to provide SEQ ID NO: 246). Other fragments omit one or more protein domains. Mutant forms of Sta006 are reported in reference 6. A Sta006 antigen may be lapidated e.g. with an acylated N-terminus cysteine. One useful Sta006 sequence is SEQ ID NO: 248, which has a Met-Ala-Ser-sequence at the N-terminus. Sta006can exist as a monomer or an oligomer (e g dimer), with Ca⁺⁺ ions favouring oligomerization. The invention can use monomers and/or oligomers of Sta006. Sta006 can be a homodimer or heterodimer with Sta011.

Sta011

The ‘Sta011’ antigen is annotated as ‘lipoprotein’. In the NCTC 8325 strain Sta011 is SAOUHSC_{—}00052 and has amino acid sequence SEQ ID NO: 47 (GI:88193872).

Sta011 antigens used with the invention can elicit an antibody (e.g. when administered to a human) that recognises SEQ ID NO: 47 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 47; and/or (b) comprising a fragment of at least ‘n’ consecutive amino acids of SEQ ID NO: 47, wherein ‘n’ is 7 or more (e.g. 8, 10, 12, 14, 16, 18, 20, 25, 30, 35, 40, 50, 60, 70, 80, 90, 100, 150, 200, 250 or more). These Sta011 proteins include variants of SEQ ID NO: 47. Preferred fragments of (b) comprise an epitope from SEQ ID NO: 47. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 47 while retaining at least one epitope of SEQ ID NO: 47. The first 23 N-terminal amino acids of SEQ ID NO: 47 can usefully be omitted (to provide SEQ ID NO: 247). Other fragments omit one or more protein domains.A Sta011 antigen may be lapidated e.g. with an acylated N-terminus cysteine. One useful Sta011 sequence is SEQ ID NO: 249, which has a N-terminus methionine. Variant forms of SEQ ID NO: 47 which may be used as or for preparing Sta011 antigens include, but are not limited to, SEQ ID NOs: 213, 214 and 215 with various Ile/Val/Leu substitutions.Sta011 can exist as a monomer or an oligomer, with Ca⁺⁺ ions favouring oligomerisation. The invention can use monomers and/or oligomers of Sta011.

Hla

The ‘Hla’ antigen is the ‘alpha-hemolysin precursor’ also known as ‘alpha toxin’ or simply ‘hemolysin’. In the NCTC 8325 strain Hla is SAOUHSC 01121 and has amino acid sequence SEQ ID NO: 14 (GI:88194865). Hla is an important virulence determinant produced by most strains of S.aureus, having pore-forming and haemolytic activity. Anti-Hla antibodies can neutralise the detrimental effects of the toxin in animal models, and Hla is particularly useful for protecting against pneumonia.

Hla antigens used with the invention can elicit an antibody (e.g. when administered to a human) that recognises SEQ ID NO: 14 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 14; and/or (b) comprising a fragment of at least ‘n’ consecutive amino acids of SEQ ID NO: 14, wherein ‘n’ is 7 or more (e.g. 8, 10, 12, 14, 16, 18, 20, 25, 30, 35, 40, 50, 60, 70, 80, 90, 100, 150, 200, 250 or more). These Hla proteins include variants of SEQ ID NO: 14. Preferred fragments of (b) comprise an epitope from SEQ ID NO: 14. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 14 while retaining at least one epitope of SEQ ID NO: 14. The first 26 N-terminal amino acids of SEQ ID NO: 14 can usefully be omitted (e.g. to give SEQ ID NO: 231). Truncation at the C-terminus can also be used e.g. leaving only 50 amino acids (residues 27-76 of SEQ ID NO: 14) [7]. Other fragments omit one or more protein domains.

Hla's toxicity can be avoided in compositions of the invention by chemical inactivation (e.g.using formaldehyde, glutaraldehyde or other cross-linking reagents). Instead, however, it is preferred to use mutant forms of Hla which remove its toxic activity while retaining its immunogenicity. Such detoxified mutants are already known in the art. One useful Hla antigen has a mutation at residue 61 of SEQ ID NO: 14, which is residue 35 of the mature antigen (i.e. after omitting the first 26 N-terminal amino acids=residue 35 of SEQ ID NO: 231). Thus residue 61 may not be histidine, and may instead be e.g.Ile, Val or preferably Leu. A His-Arg mutation at this position can also be used. For example, SEQ ID NO: 150 is the mature mutant Hla-H35L sequence (i.e. SEQ ID NO: 231 with a H35L mutation) and a useful Hla antigen comprises SEQ ID NO: 150. Another useful mutation replaces a long loop with a short sequence e.g.to replace the 39 mer at residues 136-174 of SEQ ID NO: 14 with a tetramer such as PSGS (SEQ ID NO: 225), as in SEQ ID NO: 189 (which also includes the H35L mutation) and SEQ ID NO: 216 (which does not include the H35L mutation). Another useful mutation replaces residue Y101 e.g. with a leucine (SEQ ID NO: 242). Another useful mutation replaces residue D152 e.g. with a leucine (SEQ ID NO: 243). Another useful mutant replaces residues H35 and Y101 e.g. with a leucine (SEQ ID NO: 244). Another useful mutant replaces residues H35 and D152 e.g. with a leucine (SEQ ID NO: 245).

Further useful Hla antigens are disclosed in references 8 and 9.

SEQ ID NOs: 160, 161 & 194 are three useful fragments of SEQ ID NO: 14 (‘Hla₂₇₋₇₆’, ‘Hla₂₇₋₈₉’ and ‘Hla₂₇₋₇₉’, respectively). SEQ ID NOs: 158, 159 and 195 are the corresponding fragments from SEQ ID NO: 150.

One useful Hla sequence is SEQ ID NO: 232, which was used in the examples. It has a N-terminal Met, then an Ala-Ser dipeptide from the expression vector, then SEQ ID NO: 150 (from NCTC8325 strain). It is encoded by SEQ ID NO: 233.

Hybrid Polypeptides

Antigens used in the invention may be present in the composition as individual separate polypeptides. Where more than one antigen is used, however, they do not have to be present as separate polypeptides. Instead, at least two (e.g.2, 3, 4, 5, or more) antigens can be expressed as a single polypeptide chain (a ‘hybrid’ polypeptide). Hybrid polypeptides offer two main advantages: first, a polypeptide that may be unstable or poorly expressed on its own can be assisted by adding a suitable hybrid partner that overcomes the problem; second, commercial manufacture is simplified as only one expression and purification need be employed in order to produce two polypeptides which are both antigenically useful.

The hybrid polypeptide may comprise two or more polypeptide sequences from the first antigen group. The hybrid polypeptide may comprise one or more polypeptide sequences from the first antigen group and one or more polypeptide sequences from the second antigen group. Moreover, the hybrid polypeptide may comprise two or more polypeptide sequences from each of the antigens listed above, or two or more variants of the same antigen in the cases in which the sequence has partial variability across strains.

Hybrids consisting of amino acid sequences from two, three, four, five, six, seven, eight, nine, or ten antigens are useful. In particular, hybrids consisting of amino acid sequences from two, three, four, or five antigens are preferred, such as two or three antigens.

Different hybrid polypeptides may be mixed together in a single formulation. The hybrid polypeptides can also be combined with conjugates or non-S. aureus antigens as described above.

Hybrid polypeptides can be represented by the formula NH₂-A-{-X-L-}ₙ-B—COOH, wherein: X is an amino acid sequence of a S.aureus antigen, as described above; L is an optional linker amino acid sequence; A is an optional N-terminal amino acid sequence; B is an optional C-terminal amino acid sequence; n is an integer of 2 or more (e.g.2, 3, 4, 5, 6, etc.). Usually n is 2 or 3.

If a —X— moiety has a leader peptide sequence in its wild-type form, this may be included or omitted in the hybrid protein. In some embodiments, the leader peptides will be deleted except for that of the —X— moiety located at the N-terminus of the hybrid protein i.e. the leader peptide of X₁ will be retained, but the leader peptides of X₂ . . . Xₙ will be omitted. This is equivalent to deleting all leader peptides and using the leader peptide of X₁ as moiety -A-.

For each n instances of {-X-L-}, linker amino acid sequence -L-may be present or absent. For instance, when n=2 the hybrid may be NH₂—X₁-L₁-X₂-L₂-COOH, NH₂—X₁—X₂—COOH, NH₂-X₁-L₁-X₂—COOH, NH₂—X₁—X₂-L₂-COOH, etc. Linker amino acid sequence(s) -L-will typically be short (e.g.20 or fewer amino acids i.e. 20, 19, 18, 17, 16, 15, 14, 13, 12, 11, 10, 9, 8, 7, 6, 5, 4, 3, 2, 1). Examples comprise short peptide sequences which facilitate cloning, poly-glycine linkers (i.e. comprising Glyₙ where n=2, 3, 4, 5, 6, 7, 8, 9, 10 or more), and histidine tags (i.e. His where n=3, 4, 5, 6, 7, 8, 9, 10 or more). Other suitable linker amino acid sequences will be apparent to those skilled in the art. A useful linker is GSGGGG (SEQ ID NO: 171) or GSGSGGGG (SEQ ID NO: 172), with the Gly-Ser dipeptide being formed from a BamHI restriction site (or two of them, to form the SEQ ID NO: 230 tetrapeptide), thus aiding cloning and manipulation, and the (Gly)₄ tetrapeptide(SEQ ID NO: 227) being a typical poly-glycine linker. Other suitable linkers, particularly for use as the final Lₙ are ASGGGS (SEQ ID NO: 173e.g. encoded by SEQ ID NO: 174) or a Leu-Glu dipeptide.

-A- is an optional N-terminal amino acid sequence. This will typically be short (e.g. 40 or fewer amino acids i.e. 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g.histidine tags i.e. Hisₙ where n=3, 4, 5, 6, 7, 8, 9, 10 or more). Other suitable N-terminal amino acid sequences will be apparent to those skilled in the art. If X₁ lacks its own N-terminus methionine, -A- is preferably an oligopeptide (e.g. with 1, 2, 3, 4, 5, 6, 7 or 8 amino acids) which provides a N-terminus methionine e.g. Met-Ala-Ser, or a single Met residue.

-B- is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. His, where n=3, 4, 5, 6, 7, 8, 9, 10 or more, such as SEQ ID NO: 226), or sequences which enhance protein stability. Other suitable C-terminal amino acid sequences will be apparent to those skilled in the art.

One hybrid polypeptide of the invention may include both EsxA and EsxB antigens. These may be in either order, N- to C-terminus. SEQ ID NOs: 151 (‘EsxAB’; encoded by SEQ ID NO: 169) and 152 (‘EsxBA’) are examples of such hybrids, both having hexapeptide linkers ASGGGS (SEQ ID NO: 173). Another ‘EsxAB’ hybrid comprises SEQ ID NO: 241, which may be provided with a N-terminus methionine (e.g. SEQ ID NO: 250).

Another hybrid polypeptide of the invention may include both Hla and Sta006 antigens. These may be in either order, N- to C-terminus. SEQ ID NO: 222 (‘HlaH35L-Sta006’) is an example of such a hybrid, in which a H35L mutant of Hla is joined to Sta006 via hexapeptide linker ASGGGS (SEQ ID NO: 173).

Another hybrid polypeptide of the invention may include Hla and EsxA and EsxB antigens. These may be in any order, N- to C-terminus. SEQ ID NO: 220 (‘HlaH35L-EsxAB’) is an example of such a triple hybrid, in which a H35L mutant of Hla is joined to EsxAB via linker ASGGGS (SEQ ID NO: 173). The EsxAB already includes the same linker, so SEQ ID NO: 220 includes two of these linkers. Another example of a hybrid polypeptide including Hla and EsxA and EsxB antigens is SEQ ID NO: 237 (‘HlaH35L-EsxAB’ as used in the examples), in which a H35L mutant of Hla is joined to EsxA via linker APTARG (SEQ ID NO: 239) to replace its N-terminus, then to EsxB via linker ASGGGS (SEQ ID NO: 173) to replace its N-terminus. This hybrid can be provided with a suitable N-terminal sequence such as SEQ ID NO: 240.

Another hybrid polypeptide of the invention may include Sta006 and EsxA and EsxB antigens. These may be in any order, N- to C-terminus SEQ ID NO: 223 (‘Sta006-EsxAB’) is an example of such a triple hybrid, in which Sta006 is joined to EsxAB via linker ASGGGS (SEQ ID NO: 173). The EsxAB already includes the same linker, so SEQ ID NO: 223 includes two of these linkers. Another example of a hybrid polypeptide includingSta006and EsxA and EsxB antigens is SEQ ID NO: 238 (‘Sta006-EsxAB’ as used in the examples), in which a Sta006is joined to EsxA via linker APTARG (SEQ ID NO: 239) to replace its N-terminus, then to EsxB via linker ASGGGS (SEQ ID NO: 173) to replace its N-terminus. This hybrid can be provided with a suitable N-terminal sequence such as SEQ ID NO: 240.

Usefully, these hybrid polypeptides can elicit an antibody (e.g. when administered to a human) that recognise each of the wild-type staphylococcal proteins (e.g. as shown in the sequence listing) represented in the hybrid e.g. which recognise both wild-type EsxA and wild-type EsxB, or which recognise both wild-type Hla and wild-type Sta006, or which recognise wild-type Hla and wild-type EsxA and wild-type EsxB, or which recognise wild-type Sta006 and wild-type EsxA and wild-type EsxB.

Polypeptides used with the Invention

Polypeptides used with the invention can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides used with the invention can be prepared by various means (e.g. recombinant expression, purification from cell culture, chemical synthesis, etc.). Recombinantly-expressed proteins are preferred, particularly for hybrid polypeptides.

Polypeptides used with the invention are preferably provided in purified or substantially purified form i.e. substantially free from other polypeptides (e.g. free from naturally-occurring polypeptides), particularly from other staphylococcal or host cell polypeptides, and are generally at least about 50% pure (by weight), and usually at least about 90% pure i.e. less than about 50%, and more preferably less than about 10% (e.g.5%) of a composition is made up of other expressed polypeptides. Thus the antigens in the compositions are separated from the whole organism with which the molecule is expressed.

Polypeptides used with the invention are preferably staphylococcal polypeptides.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labelling component. Also included are, for example, polypeptides containing one or more analogs of an amino acid (including, for example, unnatural amino acids, etc.), as well as other modifications known in the art. Polypeptides can occur as single chains or associated chains.

The invention provides polypeptides comprising a sequence -P-Q- or -Q-P-, wherein: -P- is an amino acid sequence as defined above and -Q- is not a sequence as defined above i.e. the invention provides fusion proteins. Where the N-terminus codon of -P- is not ATG, but this codon is not present at the N-terminus of a polypeptide, it will be translated as the standard amino acid for that codon rather than as a Met. Where this codon is at the N-terminus of a polypeptide, however, it will be translated as Met. Examples of -Q- moieties include, but are not limited to, histidine tags (i.e. His where n=3, 4, 5, 6, 7, 8, 9, 10 or more), maltose-binding protein, or glutathione-S-transferase (GST).

Although expression of the polypeptides of the invention may take place in a Staphylococcus, the invention will usually use a heterologous host for expression (recombinant expression). The heterologous host may be prokaryotic (e.g. a bacterium) or eukaryotic. It may be E. coli, but other suitable hosts include Bacillus subtilis, Vibrio cholerae, Salmonella typhi, Salmonella typhimurium, Neisseria lactamica, Neisseria cinerea, Mycobacteria (e.g. M. tuberculosis), yeasts, etc. Compared to the wild-type S.aureus genes encoding polypeptides of the invention, it is helpful to change codons to optimise expression efficiency in such hosts without affecting the encoded amino acids.

Strains and Variants

An exemplary amino acid and nucleotide sequence for the antigens described herein can easily be found in public sequence databases from the NCTC 8325 and/or Newman S.aureus strain using their GI numbers, for example, but the invention is not limited to sequences from the NCTC 8325 and Newman strains. Genome sequences of several other strains of S. aureus are available, including those of MRSA strains N315 and Mu50 [10], MW2, N315, COL, MRSA252, MSSA476, RF122, USA300 (very virulent), JH1 and JH9. Standard search and alignment techniques can be used to identify in any of these (or other) further genome sequences the homolog of any particular sequence from the Newman or NCTC 8325 strain. Moreover, the available sequences from the Newman and NCTC 8325 strains can be used to design primers for amplification of homologous sequences from other strains. Thus the invention is not limited to these two strains, but rather encompasses such variants and homologs from other strains of S.aureus, as well as non-natural variants. In general, suitable variants of a particular SEQ ID NO include its allelic variants, its polymorphic forms, its homologs, its orthologs, its paralogs, its mutants, etc.

Thus, for instance, polypeptides used with the invention may, compared to the SEQ ID NO herein, include one or more (e.g.1, 2, 3, 4, 5, 6, 7, 8, 9, etc.) amino acid substitutions, such as conservative substitutions (i.e. substitutions of one amino acid with another which has a related side chain). Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may also include one or more (e.g.1, 2, 3, 4, 5, 6, 7, 8, 9, etc.) single amino acid deletions relative to the SEQ ID NO sequences. The polypeptides may also include one or more (e.g.1, 2, 3, 4, 5, 6, 7, 8, 9, etc.) insertions (e.g. each of 1, 2, 3, 4 or 5 amino acids) relative to the SEQ ID NO sequences.

Similarly, a polypeptide used with the invention may comprise an amino acid sequence that:
is identical (i.e. 100% identical) to a sequence disclosed in the sequence listing;
shares sequence identity (e.g. 80%, 85%, 90%, 91%, 92%, 93%, 94%, 95%, 96%, 97%, 98%, 99%, 99.5% or more) with a sequence disclosed in the sequence listing;
has 1, 2, 3, 4, 5, 6, 7, 8, 9 or 10 (or more) single amino acid alterations (deletions, insertions, substitutions), which may be at separate locations or may be contiguous, as compared to the sequences of (a) or (b); and
when aligned with a particular sequence from the sequence listing using a pair wise alignment algorithm, each moving window of x amino acids from N-terminus to C-terminus (such that for an alignment that extends to p amino acids, where p>x, there are p-x+1 such windows) has at least x-y identical aligned amino acids, where: x is selected from 20, 25, 30, 35, 40, 45, 50, 60, 70, 80, 90, 100, 150, 200; y is selected from 0.50, 0.60, 0.70, 0.75, 0.80, 0.85, 0.90, 0.91, 0.92, 0.93, 0.94, 0.95, 0.96, 0.97, 0.98, 0.99; and if x-y; is not an integer then it is rounded up to the nearest integer. The preferred pair wise alignment algorithm is the Needleman-Wunsch global alignment algorithm [11], using default parameters (e.g. with Gap opening penalty=10.0, and with Gap extension penalty=0.5, using the EBLOSUM62 scoring matrix). This algorithm is conveniently implemented in the needle tool in the EMBOSS package [12].

Where hybrid polypeptides are used, the individual antigens within the hybrid (i.e. individual —X— moieties) may be from one or more strains. Where n=2, for instance, X₂ may be from the same strain as X₁ or from a different strain. Where n=3, the strains might be (i) X₁=X₂=X₃ (ii) X₁=X₂≠X₃ (iii) X₁≠X₂=X₃ (iv) X₁≠X₂≠X₃ or (v) X₁=X₃≠X₂, etc.

Within group (c), deletions or substitutions may be at the N-terminus and/or C-terminus, or may be between the two termini. Thus a truncation is an example of a deletion. Truncations may involve deletion of up to 40 (or more) amino acids at the N-terminus and/or C-terminus. N-terminus truncation can remove leader peptides e.g. to facilitate recombinant expression in a heterologous host. C-terminus truncation can remove anchor sequences e.g. to facilitate recombinant expression in a heterologous host.

In general, when an antigen comprises a sequence that is not identical to a complete S.aureus sequence from the sequence listing (e.g. when it comprises a sequence listing with <100% sequence identity thereto, or when it comprises a fragment thereof) it is preferred in each individual instance that the antigen can elicit an antibody which recognises the respective complete S. aureus sequence.

Combinations with Saccharides

The immunogenic compositions of the invention may further comprise saccharide antigens (e.g. known saccharide antigens include the exopolysaccharide of S.aureus, which is a poly-N-acetylglucosamine (PNAG), and the capsular saccharides of S.aureus, which can be e.g. from type 5, type 8 or type 336). In some embodiments a composition does not include a S.aureus saccharide antigen.

Combinations with Non-Staphylococcal Antigens

The immunogenic compositions of the invention may further comprise non-staphylococcal antigens, and in particular with antigens from bacteria associated with nosocomial infections. For example, the immunogenic composition may further comprise one or more antigen(s) selected from the group consisting of: Clostridium difficile; Pseudomonas aeruginosa; Candida albicans; and extraintestinal pathogenic Escherichia coli. Further suitable antigens for use in combination with staphylococcal antigens of the invention are listed on pages 33-46 of reference 13.

Preferred Compositions

A preferred composition of the invention includes all four of: (i) a single polypeptide including both an EsxA antigen and an EsxB antigen e.g. comprising SEQ ID NO: 250; (ii) a Sta006 antigen e.g. comprising SEQ ID NO: 248; (iii) a Sta011 antigen e.g. comprising SEQ ID NO: 249; and (iv) a H35L mutant form of Hla e.g. comprising SEQ ID NO: 232. This composition is particularly useful when using TLR7 agonists of formula (K).

Although SEQ ID NOs: 250, 248, 249 and 232 are useful amino acid sequences in a combination, the invention is not limited to these precise sequences. Thus 1, 2, 3 or all 4 of these sequences can independently be modified by up to 5 single amino changes (i.e. 1, 2, 3, 4 or 5 single amino acid substitutions, deletions and/or insertions) provided that the modified sequence can elicit antibodies which still bind to a polypeptide consisting of the unmodified sequence.

One useful composition of the invention includes all four of: (i) a first polypeptide having amino acid sequence SEQ ID NO: 250; (ii) a second polypeptide having amino acid sequence SEQ ID NO: 248; (iii) a third polypeptide having amino acid sequence SEQ ID NO: 249; and (iv) a fourth polypeptide having amino acid sequence SEQ ID NO: 232. In some embodiments the composition may include one or more further polypeptides; in other embodiments the only polypeptides in the composition are these four specified polypeptides. SEQ ID NOs: 250, 248, 249 and 232 are useful amino acid sequences in a combination, but the invention is not limited to these precise sequences. Thus 1, 2, 3 or all 4 of these four sequences can independently be modified by 1, 2, 3, 4 or 5 single amino changes (i.e. 1, 2, 3, 4 or 5 single amino acid substitutions, deletions and/or insertions) provided that the modified sequence can elicit antibodies which still bind to a polypeptide consisting of the unmodified sequence. In a preferred embodiment, the composition thus includes these four specified polypeptides with 1, 2, 3 or all 4 of SEQ ID NO: 250, 248, 249 and 232 independently modified by 1 single amino acid substitution, deletion and/or insertion.

The four polypeptides may be present at substantially equal masses i.e. the mass of each of them is within ±5% of the mean mass of all the polypeptides. Thus they may be present at a mass ratio of a:b:c:d, where each of a-d is between 0.95 and 1.05.

Stabilizing Additives

In some embodiments of the invention an immunogenic composition includes a stabilizing additive. Such additives include, but are not limited to, chelators of divalent metal cations (e.g. EDTA, ethylenediaminetetraacetic acid), sugars (e.g. disaccharides such as sucrose or trehalose), sugar alcohols (e g. mannitol), free amino acids (e.g. arginine), buffer salts (e.g. phosphate, citrate), polyols (e.g. glycerol, mannitol), or protease inhibitors.

EDTA is a preferred additive. The final concentration of EDTA in the immunogenic composition of the invention can be about 1-50 mM, about 1-10 mM or about 1-5 mM, preferably about 2.5 mM and more preferably about 5.0 mM.

A buffer is another useful additive, in order to control pH of a composition. This can be particularly important after reconstitution of lyophilized material. Compositions of the invention may include one or more buffer(s). Typical buffers include: a phosphate buffer; a Tris buffer; a borate buffer; a succinate buffer; a histidine buffer; or a citrate buffer. A phosphate buffer is preferable. Buffers will typically be included in the 5-20 mM range. Aqueous compositions of the invention preferably have a pH of between 5 and 6.5 e.g. between 5.8-6.2, or 5.9-6.1, or a pH of 6.

A saccharide or sugar alcohol (or mixture thereof e.g. a mannitol/sucrose mixture) is also useful, particularly when using lyophilization. Suitable materials include, but are not limited to, mannitol, lactose, sucrose, trehalose, dextrose, etc. The use of sucrose is particularly preferred. Such materials can be present at a concentration of about 1% by weight per volume, or about 3% to about 6% by weight per volume, or up to about 10% or about 12.5% by weight per volume, preferably about 5% by weight per volume.

Lyophilization

One way of storing immunogenic compositions of the invention is in lyophilized form. This procedure can be used with or without the addition of a metal chelator (e.g. EDTA). The inventors have also shown that EDTA does not have a significant impact on the thermal characteristic of the vaccine and does not introduce any undesired plasticizing effect, thus meaning that EDTA-containing compositions can be lyophilized to further enhance storage stability.

Thus, generally, the invention also provides a lyophilizate which comprises a divalent metal cation chelator (e.g. EDTA) and at least one antigen (e.g. at least one polypeptide antigen).

The invention also provides a lyophilizate of an aqueous immunogenic composition of the invention. This is prepared by lyophilising an aqueous composition of the invention. It can then be reconstituted with aqueous material to provide an aqueous immunogenic composition of the invention. Materials present in the material which is lyophilized will remain in the lyophilizate and will thus also be present after reconstitution e.g. buffer salts, lyoprotectants (e.g. sucrose and/or mannitol), chelators, etc. If the material is reconstituted with a smaller volume of material than before lyophilization then these materials will be present in more concentrated form. The reconstituted lyophilizate preferably contains lyoprotectants (e.g. sucrose and/or mannitol) at a concentration of up to about 2.5% by weight per volume, preferably about 1% to about 2% by weight per volume. The amount of EDTA which is present in a composition after lyophilisation and prior to reconstitution is ideally at least 0.75 mM, and preferably at least 2.5 mM. A maximum of 50 mM is envisaged.

Liquid materials useful for reconstituting lyophilizates include, but are not limited to: salt solutions, such as physiological saline; buffers, such as PBS; water, such as wfi. They usefully have a pH between 4.5 and 7.5 e.g. between 6.8 and 7.2. The reconstituted lyophilizate preferably has a pH of between 5-6.5e.g. between 5.8-6.2, or 5.9-6.1, or a pH of 6.A liquid material for reconstitution can include an adjuvant e.g. an aluminium salt adjuvant. Aqueous suspensions of adjuvants (optionally including buffers, such as a histidine buffer) are useful for simultaneously reconstituting and adsorbing lyophilized polypeptides. In other embodiments the liquid material is adjuvant-free. Typically the lyophilizate does not include an insoluble metal salt adjuvant.

The invention also provides a lyophilizate which comprises EDTA and at least one antigen.

Immunogenic Compositions and Medicaments

Immunogenic compositions of the invention may be useful as vaccines. Vaccines according to the invention may either be prophylactic (i.e. to prevent infection) or therapeutic (i.e. to treat infection), but will typically be prophylactic.

Compositions may thus be pharmaceutically acceptable. They will usually include components in addition to the antigens e.g. they typically include one or more pharmaceutical carrier(s) and/or excipient(s). A thorough discussion of such components is available in reference 39.

Compositions will generally be administered to a mammal in aqueous form. Prior to administration, however, the composition may have been in a non-aqueous form. For instance, although some immunogenic compositions are manufactured in aqueous form, then filled and distributed and administered also in aqueous form, other immunogenic compositions are lyophilized during manufacture and are reconstituted into an aqueous form at the time of use. Thus a composition of the invention may be dried, such as a lyophilized formulation.

Where a composition of the invention includes more than one polypeptide, the mass of each different polypeptide can be the same or different. Ideally they are present at substantially equal masses i.e. the mass of each of them is within +5% of the mean mass of all the polypeptides. In embodiments where two antigens are present as a hybrid polypeptide, the hybrid is considered as a single polypeptide for this purpose. The factors that can influence the amount of the polypeptide to be included in a multivalent formulation include the amount of polypeptide sufficient to elicit an immune response and the amount that would cause aggregation (with itself or with other polypeptide) or influence the stability of the other polypeptide. Typical masses of a polypeptide in an immunogenic composition are between 1-100 m.

The composition may include preservatives such as thiomersal or 2-phenoxyethanol. It is preferred, however, that the immunogenic compositions should be substantially free from (i.e. less than 5 μg/ml) mercurial material e.g. thiomersal-free. Compositions containing no mercury are more preferred. Preservative-free compositions are particularly preferred.

To improve thermal stability, a composition may include a temperature protective agent. Further details of such agents are provided below. To control tonicity, it is preferred to include a physiological salt, such as a sodium salt. Sodium chloride (NaCl) is preferred, which may be present at between 1 and 20 mg/ml e.g. about 10±2 mg/ml NaCl. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, preferably between 240-360 mOsm/kg, and will more preferably fall within the range of 290-310 mOsm/kg.

Compositions may include one or more buffers. Typical buffers include: a phosphate buffer; a Tris buffer; a borate buffer; a succinate buffer; a histidine buffer (particularly with an aluminium hydroxide adjuvant); or a citrate buffer. Buffers will typically be included in the 5-20 mM range. The buffer is preferably 10 mM potassium phosphate.

The pH of the compositions are preferably between about 5 and about 6.5, and more preferably between about 5.5 and about 6, and most preferably at about 6.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunisation, or may include material for multiple immunisations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Human vaccines are typically administered in a dosage volume of about 0.5 ml, although a half dose (i.e. about 0.25 ml) may be administered to children.

Immunogenic compositions of the invention may also comprise one or more immunoregulatory agents. Preferably, one or more of the immunoregulatory agents include one or more adjuvants. The adjuvants may include a TH1 adjuvant and/or a TH2 adjuvant, further discussed below. Thus the immunogenic compositions may further comprise an adjuvant, such as an aluminium salt adjuvant (for example, one or more antigens may be adsorbed to aluminium salt). More generally, adjuvants which may be used in compositions of the invention include, but are not limited to, those already listed in reference 4. These include mineral-containing adjuvants and oil-in-water emulsions.

Mineral-Containing Adjuvants

Mineral containing adjuvants include mineral salts such as aluminium salts and calcium salts (or mixtures thereof). Preferably, the composition contains an aluminium salt adjuvant. Aluminium salts include hydroxides, phosphates, etc., with the salts taking any suitable form (e.g. gel, crystalline, amorphous, etc.). Calcium salts include calcium phosphate (e.g. the “CAP” particles disclosed in ref 14). Adsorption to these salts is preferred (e.g. all antigens may be adsorbed). The mineral containing compositions may also be formulated as a particle of metal salt [15].

The adjuvants known as aluminium hydroxide and aluminium phosphate may be used. These names are conventional, but are used for convenience only, as neither is a precise description of the actual chemical compound which is present (e.g.see chapter 9 of referencel6)). The invention can use any of the “hydroxide” or “phosphate” adjuvants that are in general use as adjuvants. The adjuvants known as “aluminium hydroxide” are typically aluminium oxyhydroxide salts, which are usually at least partially crystalline. The adjuvants known as “aluminium phosphate” are typically aluminium hydroxyphosphates, often also containing a small amount of sulphate (i.e. aluminium hydroxyphosphatesulphate). They may be obtained by precipitation, and the reaction conditions and concentrations during precipitation influence the degree of substitution of phosphate for hydroxyl in the salt.

A fibrous morphology (e.g. as seen in transmission electron micrographs) is typical for aluminium hydroxide adjuvants. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

Aluminium phosphate adjuvants generally have a PO₄/Al molar ratio between 0.3 and 1.2, preferably between 0.8 and 1.2, and more preferably 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg A³⁺/ml. The aluminium phosphate will generally be particulate (e.g. plate-like morphology as seen in transmission electron micrographs). Typical diameters of the particles are in the range 0.1-10 μm (e.g. about 0.1-5 μm) after any antigen adsorption. Adsorptive capacities of between 0.7-1.5 mg protein per mg Al⁺⁺⁺ at pH 7.4 have been reported for aluminium phosphate adjuvants.

The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5 and 6.5 e.g. about 5.7.

Suspensions of aluminium salts used to prepare compositions of the invention may contain a buffer (e.g. a phosphate or a histidine or a Tris buffer), but this is not always necessary. The suspensions are preferably sterile and pyrogen-free. A suspension may include free aqueous phosphate ions e.g. present at a concentration between 1.0 and 20 mM, preferably between 5 and 15 mM, and more preferably about 10 mM. The suspensions may also comprise sodium chloride.

The preferred aluminium salt adjuvant is an aluminium hydroxide adjuvant.

The invention can use a mixture of both an aluminium hydroxide and an aluminium phosphate. In this case there may be more aluminium phosphate than hydroxide e.g. a weight ratio of at least 2:1 e.g. ≧5:1, ≧6:1, ≧7:1, ≧8:1, ≧9:1, etc.

The concentration of Al⁺⁺⁺ in a composition for administration to a patient is preferably less than 10 mg/ml e.g. ≦5 mg/ml,≦4 mg/ml, ≦3 mg/ml, ≦2 mg/ml, ≦1 mg/ml, etc. A preferred range is between 0.3 and 1 mg/ml. A maximum of 0.85 mg/dose is preferred.

A mineral salt can usefully have a TLR agonist, such as a TLR7 agonist, adsorbed to it (e.g. see ref 17).

Oil & Water Emulsions

Oil emulsion compositions suitable for use as adjuvants in the invention include oil-in-water emulsions such as MF59 (Chapter 10 of ref 16; see also ref 18) and AS03. Complete Freund's adjuvant (CFA) and incomplete Freund's adjuvant (IFA) may also be used.

Various oil-in-water emulsion adjuvants are known, and they typically include at least one oil and at least one surfactant, with the oil(s) and surfactant(s) being biodegradable (metabolisable) and biocompatible. The oil droplets in the emulsion are generally less than 5 μm in diameter, and ideally have a sub-micron diameter, with these small sizes being achieved with a microfluidiser to provide stable emulsions. Droplets with a size less than 220 nm are preferred as they can be subjected to filter sterilization.

The emulsion can comprise oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g.obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoids known as squalene, 2,6,10,15,19,23-hexamethyl-2,6,10,14,18,22-tetracosahexaene, which is particularly preferred herein. Squalane, the saturated analog to squalene, is also a preferred oil. Fish oils, including squalene and squalane, are readily available from commercial sources or may be obtained by methods known in the art. Other preferred oils are the tocopherols (see below). Mixtures of oils can be used.

Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylenesorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenolethoxylates, such as the Tergitol™ NP series; polyoxyethylene fatty ethers derived from lauryl, cetyl, stearyl and oleyl alcohols (known as Brij surfactants), such as triethyleneglycolmonolauryl ether (Brij 30); and sorbitan esters (commonly known as the SPANs), such as sorbitantrioleate (Span 85) and sorbitanmonolaurate. Non-ionic surfactants are preferred. Preferred surfactants for including in the emulsion are Tween 80 (polyoxyethylenesorbitanmonooleate), Span 85 (sorbitantrioleate), lecithin and Triton X-100.

Mixtures of surfactants can be used e.g. Tween 80/Span 85 mixtures. A combination of a polyoxyethylenesorbitan ester such as polyoxyethylenesorbitanmonooleate (Tween 80) and an octoxynol such as t-octylphenoxypolyethoxyethanol (Triton X-100) is also suitable. Another useful combination comprises laureth 9 plus a polyoxyethylenesorbitan ester and/or an octoxynol.

Preferred amounts of surfactants (% by weight) are: polyoxyethylenesorbitan esters (such as Tween 80) 0.01 to 1%, in particular about 0.1%; octyl- or nonylphenoxypolyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Preferred emulsion adjuvants have an average droplets size of <1 μm e.g. ≦750 nm, ≦500 nm, ≦400 nm, ≦300 nm, ≦250 nm, ≦220 nm, ≦200 nm, or smaller. These droplet sizes can conveniently be achieved by techniques such as microfluidisation.

Specific oil-in-water emulsion adjuvants useful with the invention include, but are not limited to:
A submicron emulsion of squalene, polysorbate 80, and sorbitantrioleate. These three components can be present at a volume ratio of 10:1:1 or a weight ratio of 39:47:47. The composition of the emulsion by volume can be about 5% squalene, about 0.5% polysorbate 80 and about 0.5% sorbitantrioleate. In weight terms, these ratios become 4.3% squalene, 0.5% polysorbate 80 and 0.48% sorbitantrioleate. This adjuvant is known as ‘MF59’ [19-21], as described in more detail in Chapter 10 of ref.22and chapter 12 of ref. 23. The MF59 emulsion advantageously includes citrate ions e.g. 10 mM sodium citrate buffer.
An emulsion of squalene, a tocopherol, and polysorbate 80. The emulsion may include phosphate buffered saline. It may also include Span 85 (e.g. at 1%) and/or lecithin. These emulsions may have from 2 to 10% squalene, from 2 to 10% tocopherol and from 0.3 to 3% polysorbate 80, and the weight ratio of squalene:tocopherol is preferably ≦1 as this provides a more stable emulsion. Squalene and polysorbate 80 may be present volume ratio of about 5:2 or at a weight ratio of about 11:5. Thus the three components (squalene, tocopherol, polysorbate 80) may be present at a weight ratio of 1068:1186:485 or around 55:61:25. One such emulsion (‘AS03’) can be made by dissolving Tween 80 in PBS to give a 2% solution, then mixing 90 ml of this solution with a mixture of (5 g of DL-α-tocopherol and 5 ml squalene), then microfluidising the mixture. The resulting emulsion may have submicron oil droplets e.g. with an average diameter of between 100 and 250 nm, preferably about 180 nm. The emulsion may also include a 3-de-O-acylated monophosphoryl lipid A (3d-MPL). Another useful emulsion of this type may comprise, per human dose, 0.5-10 mg squalene, 0.5-11 mg tocopherol, and 0.1-4 mg polysorbate 80 [24] e.g. in the ratios discussed above.
An emulsion of squalene, a tocopherol, and a Triton detergent (e.g. Triton X-100). The emulsion may also include a 3d-MPL (see below). The emulsion may contain a phosphate buffer.
An emulsion comprising a polysorbate (e.g.polysorbate 80), a Triton detergent (e.g. Triton X-100) and a tocopherol (e.g.an α-tocopherol succinate). The emulsion may include these three components at a mass ratio of about 75:11:10 (e.g.750 m/ml polysorbate 80, 110 m/ml Triton X-100 and 100 μm/ml α-tocopherol succinate), and these concentrations should include any contribution of these components from antigens. The emulsion may also include squalene. The emulsion may also include a 3d-MPL (see below). The aqueous phase may contain a phosphate buffer.
An emulsion of squalene, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant [25] (0.05-1% Thr-MDP, 5% squalane, 2.5% Pluronic L121 and 0.2% polysorbate 80). It can also be used without the Thr-MDP, as in the “AF” adjuvant [26] (5% squalane, 1.25% Pluronic L121 and 0.2% polysorbate 80). Microfluidisation is preferred.
An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. asorbitan ester or mannide ester, such as sorbitanmonoleate or ‘Span 80’). The emulsion is preferably thermoreversible and/or has at least 90% of the oil droplets (by volume) with a size less than 200 nm [27]. The emulsion may also include one or more of: alditol; a cryoprotective agent (e.g.a sugar, such as dodecylmaltoside and/or sucrose); and/or an alkylpolyglycoside. The emulsion may include a TLR4 agonist [28]. Such emulsions may be lyophilized.
An emulsion of squalene, poloxamer 105 and Abil-Care [29]. The final concentration (weight) of these components in adjuvanted vaccines are 5% squalene, 4% poloxamer 105 (pluronicpolyol) and 2% Abil-Care 85 (Bis-PEG/PPG-16/16 PEG/PPG-16/16 dimethicone; caprylic/capric triglyceride).
An emulsion having from 0.5-50% of an oil, 0.1-10% of a phospholipid, and 0.05-5% of a non-ionic surfactant. As described in reference 30, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilAsaponin, cholesterol, a saponin-lipophile conjugate (such as GPI-0100, described in reference 31, produced by addition of aliphatic amine to desacylsaponin via the carboxyl group of glucuronic acid), dimethyidioctadecylammonium bromide and/or N,N-dioctadecyl-N,N-bis(2-hydroxyethyl)propanediamine
An emulsion in which a saponin (e.g. QuilA or QS21) and a sterol (e.g. a cholesterol) are associated as helical micelles [32].
An emulsion comprising a mineral oil, a non-ionic lipophilic ethoxylated fatty alcohol, and a non-ionic hydrophilic surfactant (e.g. an ethoxylated fatty alcohol and/or polyoxyethylene-polyoxypropylene block copolymer) [33].
An emulsion comprising a mineral oil, a non-ionic hydrophilic ethoxylated fatty alcohol, and a non-ionic lipophilic surfactant (e.g. an ethoxylated fatty alcohol and/or polyoxyethylene-polyoxypropylene block copolymer) [33].

In some embodiments an emulsion may be mixed with antigen extemporaneously, at the time of delivery, and thus the adjuvant and antigen may be kept separately in a packaged or distributed composition, ready for final formulation at the time of use. In other embodiments an emulsion is mixed with antigen during manufacture, and thus the composition is packaged in a liquid adjuvanted form,. The antigen will generally be in an aqueous form, such that the composition is finally prepared by mixing two liquids. The volume ratio of the two liquids for mixing can vary (e.g. between 5:1 and 1:5) but is generally about 1:1. Where concentrations of components are given in the above descriptions of specific emulsions, these concentrations are typically for an undiluted composition, and the concentration after mixing with an antigen solution will thus decrease.

Where a composition includes a tocopherol, any of the α, β, γ, δ, ε or ξtocopherols can be used, but α-tocopherols are preferred. The tocopherol can take several forms e.g. different salts and/or isomers. Salts include organic salts, such as succinate, acetate, nicotinate, etc. D-α-tocopherol and DL-α-tocopherol can both be used. Tocopherols are advantageously included in compositions for use in elderly patients (e.g. aged 60 years or older) because vitamin E has been reported to have a positive effect on the immune response in this patient group [34]. They also have antioxidant properties that may help to stabilize the emulsions [35]. A preferred α-tocopherol is DL-α-tocopherol, and the preferred salt of this tocopherol is the succinate.

The use of an aluminium hydroxide and/or aluminium phosphate adjuvant is particularly preferred, and antigens are generally adsorbed to these salts.

Compositions of the invention may elicit both a cell mediated immune response as well as a humoral immune response. This immune response will preferably induce long lasting (e.g. neutralising) antibodies and a cell mediated immunity that can quickly respond upon exposure to S. aureus.

The immune response may be one or both of a TH1 immune response and a TH2 response. Preferably, immune response provides for one or both of an enhanced TH1 response and an enhanced TH2 response.

The enhanced immune response may be one or both of a systemic and a mucosal immune response. Preferably, the immune response provides for one or both of an enhanced systemic and an enhanced mucosal immune response. Preferably the mucosal immune response is a TH2 immune response. Preferably, the mucosal immune response includes an increase in the production of IgA.

S. aureus infections can affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared (e.g.a lyophilized composition or a spray-freeze dried composition). The composition may be prepared for topical administration e.g.as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilized antigens.

Where a composition is to be prepared extemporaneously prior to use (e.g.where a component is presented in lyophilized form) and is presented as a kit, the kit may comprise two vials, or it may comprise one ready-filled syringe and one vial, with the contents of the syringe being used to reactivate the contents of the vial prior to injection.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. Where more than one antigen is included in a composition then two antigens may be present at the same dose as each other or at different doses.

As mentioned above, a composition may include a temperature protective agent, and this component may be particularly useful in adjuvanted compositions (particularly those containing a mineral adjuvant, such as an aluminium salt). As described in reference 36, a liquid temperature protective agent may be added to an aqueous vaccine composition to lower its freezing point e.g.to reduce the freezing point to below 0° C. Thus the composition can be stored below 0° C., but above its freezing point, to inhibit thermal breakdown. The temperature protective agent also permits freezing of the composition while protecting mineral salt adjuvants against agglomeration or sedimentation after freezing and thawing, and may also protect the composition at elevated temperatures e.g.above 40° C. A starting aqueous vaccine and the liquid temperature protective agent may be mixed such that the liquid temperature protective agent forms from 1-80% by volume of the final mixture. Suitable temperature protective agents should be safe for human administration, readily miscible/soluble in water, and should not damage other components (e.g. antigen and adjuvant) in the composition. Examples include glycerin, propylene glycol, and/or polyethylene glycol (PEG). Suitable PEGs may have an average molecular weight ranging from 200-20,000 Da. In a preferred embodiment, the polyethylene glycol can have an average molecular weight of about 300 Da (‘PEG-300’).

Methods of Treatment, and Administration of the Vaccine

The invention also provides a method for raising an immune response in a mammal comprising the step of administering a composition of the invention to the mammal The immune response is preferably protective and preferably involves antibodies and/or cell-mediated immunity The method may raise a booster response.

The invention also provides the use of an EsxA antigen, an EsxB antigen and a stabilizing additive, in the manufacture of a medicament for raising an immune response in a mammal The use may also involve a Sta006 antigen, a Sta011 antigen and/or a Hla antigen. It may also involve the use of an adjuvant.

The invention also provides the use of an EsxAB antigen and a stabilizing additive, in the manufacture of a medicament for raising an immune response in a mammal The use may also involve a Sta006 antigen, a Sta011 antigen and/or a Hla antigen.It may also involve the use of an adjuvant.

The invention also provides the use of EDTA and an antigen in the manufacture of a lyophilized 25 medicament for raising, after reconstitution, an immune response in a mammal

By raising an immune response in the mammal by these uses and methods, the mammal can be protected against S.aureus infection, including a nosocomial infection. More particularly, the mammal may be protected against a skin infection, pneumonia, meningitis, osteomyelitis endocarditis, toxic shock syndrome, and/or septicaemia.

The invention also provides a kit comprising a first component and a second component wherein neither the first component nor the second component is a composition of the invention as described above, but wherein the first component and the second component can be combined to provide a composition of the invention as described above. The kit may further include a third component comprising one or more of the following: instructions, syringe or other delivery device, adjuvant, or pharmaceutically acceptable formulating solution.

The invention also provides a delivery device pre-filled with an immunogenic composition of the invention.

The mammal is preferably a human. Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g.to assess safety, dosage, immunogenicity, etc. Other mammals which can usefully be immunised according to the invention are cows, dogs, horses, and pigs.

One way of checking efficacy of therapeutic treatment involves monitoring S.aureus infection after administration of the compositions of the invention. One way of checking efficacy of prophylactic treatment involves monitoring immune responses, systemically (such as monitoring the level of IgG1 and IgG2a production) and/or mucosally (such as monitoring the level of IgA production), against the antigens in the compositions of the invention after administration of the composition. Typically, antigen-specific serum antibody responses are determined post-immunisation but pre-challenge whereas antigen-specific mucosal antibody responses are determined post-immunisation and post-challenge.

Another way of assessing the immunogenicity of the compositions of the present invention is to express the proteins recombinantly for screening patient sera or mucosal secretions by immunoblot and/or microarrays. A positive reaction between the protein and the patient sample indicates that the patient has mounted an immune response to the protein in question. This method may also be used to identify immunodominant antigens and/or epitopes within antigens.

The efficacy of immunogenic compositions can also be determined in vivo by challenging animal models of S.aureus infection, e.g., guinea pigs or mice, with the immunogenic compositions. In particular, there are three useful animal models for the study of S.aureus infectious disease, namely: (i) the murine abscess model [37], (ii) the murine lethal infection model [37] and (iii) the murine pneumonia model [38]. The abscess model looks at abscesses in mouse kidneys after intravenous challenge. The lethal infection model looks at the number of mice which survive after being infected by a normally-lethal dose of S.aureus by the intravenous or intraperitoneal route. The pneumonia model also looks at the survival rate, but uses intranasal infection. A useful immunogenic composition may be effective in one or more of these models. For instance, for some clinical situations it may be desirable to protect against pneumonia, without needing to prevent hematic spread or to promote opsonisation; in other situations the main desire may be to prevent hematic spread. Different antigens, and different antigen combinations, may contribute to different aspects of an effective immunogenic composition.

Compositions of the invention will generally be administered directly to a patient. Direct delivery may be accomplished by parenteral injection (e.g. subcutaneously, intraperitoneally, intravenously, intramuscularly, or to the interstitial space of a tissue), or mucosally, such as by rectal, oral (e.g.tablet, spray), vaginal, topical, transdermal or transcutaneous, intranasal, ocular, aural, pulmonary or other mucosal administration.

The invention may be used to elicit systemic and/or mucosal immunity, preferably to elicit an enhanced systemic and/or mucosal immunity

Preferably the enhanced systemic and/or mucosal immunity is reflected in an enhanced TH1 and/or TH2 immune response. Preferably, the enhanced immune response includes an increase in the production of IgG1 and/or IgG2a and/or IgA.

Dosage can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Multiple doses will typically be administered at least 1 week apart (e.g.about 2 weeks, about 3 weeks, about 4 weeks, about 6 weeks, about 8 weeks, about 10 weeks, about 12 weeks, about 16 weeks, etc.).

Vaccines prepared according to the invention may be used to treat both children and adults. Thus a human patient may be less than 1 year old, 1-5 years old, 5-15 years old, 15-55 years old, or at least 55 years old. Preferred patients for receiving the vaccines are the elderly (e.g.≧50 years old, ≧60 years old, and preferably ≧65 years), the young (e.g. ≦5 years old), hospitalised patients, healthcare workers, armed service and military personnel, pregnant women, the chronically ill, or immunodeficient patients. The vaccines are not suitable solely for these groups, however, and may be used more generally in a population.

Vaccines produced by the invention may be administered to patients at substantially the same time as (e.g. during the same medical consultation or visit to a healthcare professional or vaccination centre) other vaccines e.g. at substantially the same time as an influenza vaccine, a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H.influenzae type b vaccine, an inactivated poliovirus vaccine, a hepatitis B virus vaccine, a meningococcal conjugate vaccine (such as a tetravalent A-C-W135-Y vaccine), a respiratory syncytial virus vaccine, etc. Further non-staphylococcal vaccines suitable for co-administration may include one or more antigens listed on pages 33-46 of reference 13.

General

The practice of the present invention will employ, unless otherwise indicated, conventional methods of chemistry, biochemistry, molecular biology, immunology and pharmacology, within the skill of the art. Such techniques are explained fully in the literature. See, e.g., references 39-46, etc.

“GI” numbering is used above. A GI number, or “Genlnfo Identifier”, is a series of digits assigned consecutively to each sequence record processed by NCBI when sequences are added to its databases. The GI number bears no resemblance to the accession number of the sequence record. When a sequence is updated (e.g. for correction, or to add more annotation or information) then it receives a new GI number. Thus the sequence associated with a given GI number is never changed.

Where the invention concerns an “epitope”, this epitope may be a B-cell epitope and/or a T-cell epitope. Such epitopes can be identified empirically (e.g.using PEPSCAN [47,48] or similar methods), or they can be predicted (e.g.using the Jameson-Wolf antigenic index [49], matrix-based approaches [50], MAPITOPE [51], TEPITOPE [52,53], neural networks [54], OptiMer&EpiMer [55, 56], ADEPT [57], Tsites [58], hydrophilicity [59], antigenic index [60] or the methods disclosed in references 61-65, etc.).

Epitopes are the parts of an antigen that are recognised by and bind to the antigen binding sites of antibodies or T-cell receptors, and they may also be referred to as “antigenic determinants”.

Where an antigen “domain” is omitted, this may involve omission of a signal peptide, of a cytoplasmic domain, of a transmembrane domain, of an extracellular domain, etc.

The term “comprising” encompasses “including” as well as “consisting” e.g. a composition “comprising” X may consist exclusively of X or may include something additional e.g.X+Y.

The term “about” in relation to a numerical value x is optional and means, for example, x±10%.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of ref 66. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref 67.

### BRIEF DESCRIPTION OF DRAWINGS

FIG. 1 shows Capillary Electrophoresis profiles of the (A) monovalent (EsxAB), (B) trivalent (Sta006, Sta011 and HlaH35L) and (C) tetravalent (EsxAB, Sta006, Sta011 and HlaH35L)pre-lyophilized formulation at pH6 in the absence of EDTA incubated at 25° C. for up to 72 hours. Peaks: EsxAB monomer (15 min), Sta011 monomer (17 min), HlaH35L (18 min), EsxAB dimer (19 min), Sta011 dimer (21 min), Sta006 dimer (21.5 min)

FIG. 2 shows the Differential Scanning calorimetry profile of pre-lyophilized formulation in 0.75 mM EDTA (glass transition temperature (Tg) at −33.39° C.).

FIG. 3 shows the differential scanning calorimetry profile of pre-lyophilized formulation in 5 mM EDTA (glass transition temperature (Tg) at −32.86° C.).

FIG. 4 shows the size exclusion chromatography profile of the tetravalent (EsxAB, Sta006, Sta011 and HlaH35L)pre-lyophilized formulation in 0.75 mM EDTA incubated at 2-8° C. for up to 72 hours. Peaks: Sta006+Sta011+EsxAB (11 min), HlaH35L (12.5 min), EDTA (14 min)

FIG. 5 shows the size exclusion chromatography profile of the tetravalent (EsxAB, Sta006, Sta011 and HlaH35L)pre-lyophilized formulation in 0.75 mM EDTA incubated at 25° C. for up to 72 hours. Peaks: Sta006+Sta011+EsxAB (11 min), HlaH35L (12.5 min), EDTA (14 min)

FIG. 6 shows the size exclusion chromatography profile of the tetravalent (EsxAB, Sta006, Sta011 and HlaH35L)pre-lyophilized formulation in 5 mM EDTA incubated at 2-8° C. for up to 72 hours. Peaks: Sta006+Sta011+EsxAB (11 min), HlaH35L (12.5 min), EDTA (14 min)

FIG. 7 shows the size exclusion chromatography profile of the tetravalent (EsxAB, Sta006, Sta011 and HlaH35L)pre-lyophilized formulation in 5 mM EDTA incubated at 25° C. for up to 72 hours. Peaks: Sta006+Sta011+EsxAB (11 min), HlaH35L (12.5 min), EDTA (14 min)

FIG. 8 shows native protein gels of the monovalent and tetravalent pre-lyophilized formulations in5 mM EDTA incubated at 2-8° C. and 25° C. at t=0 (A), after 24 hours (B), after 48 hours (C) and after 72 hours (D).Lane 1: Sta006; Lane 2: Sta011; Lane 3: HlaH35; Lane 4: EsxAB; Lane 5: EsxAB, Sta006, Sta011 and HlaH35L.MW: EsxAB monomer (22.8 kDa), Sta011 monomer(27 kDa), Sta006 monomer(32 kDa), HlaH35L (33 kDa), EsxAB dimer (45.6 kDa), Sta011 dimer (54 kDa), Sta006 dimer(64 kDa), EsxAB dimer (91.2 kDa). MW markers.

FIG. 9shows Capillary Electrophoresis profiles of the tetravalent (EsxAB, Sta006, Sta011 and HlaH35L) pre-lyophilized formulation at (A) pH7.2 and (B) pH 6 incubated at room temperature for up to 72 hours.Peaks: EsxAB monomer (15 min), Sta011 monomer (17 min), HlaH35L (18 min), EsxAB dimer (19 min), Sta011 dimer (21 min), Sta006 dimer (21.5 min)

FIG. 10 shows the protein selectivity of monomer to dimer as a function of pH for Sta006, Sta011 and EsxAB, recorded by reverse phase chromatography.Sta006 (♦), Sta011 (▪), EsxAB (▴).

FIG. 11 shows the size exclusion chromatography profiles of the tetravalent (EsxAB, Sta006, Sta011 and HlaH35L) (A) pre-lyophilized formulation formulation in 5 mM EDTA at pH6 incubated at room temperature for 24 hours and (B) lyophilized formulation reconstituted in aqueous solution.

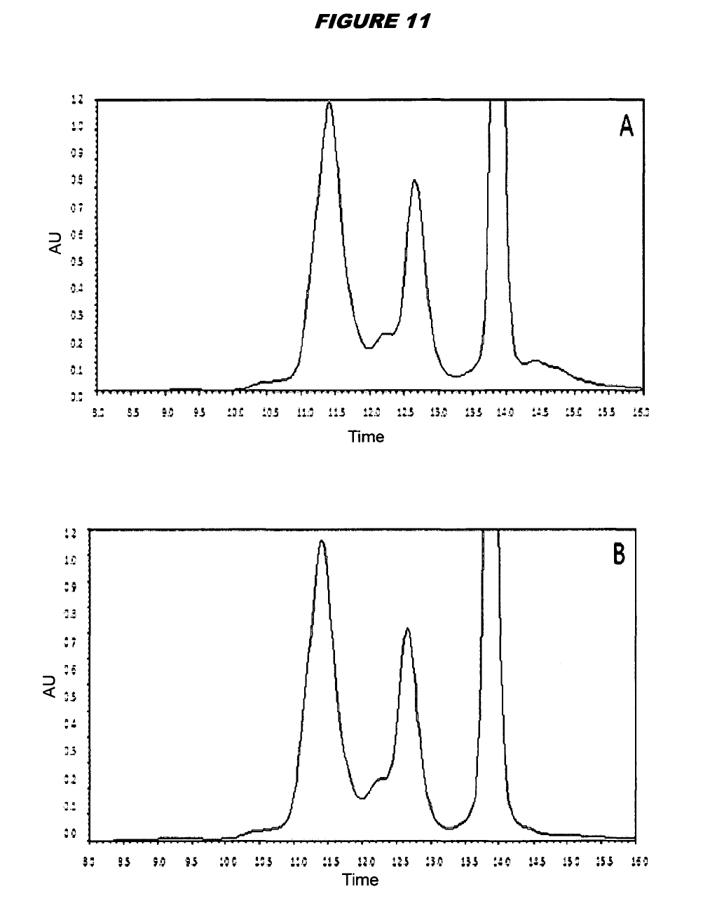

FIG. 12 shows the antibody titres against the HlaH35L antigen in mice following immunization with reconstituted lyophilized adjuvanted tetravalent vaccines that had been prepared in pre-lyophilization formulations at pH 6.0 or pH 7.2. Controls received identical courses of saline plus adjuvant.

FIG. 13 shows the antibody titres against the Sta006 antigen in mice following immunization with reconstituted lyophilized adjuvanted tetravalent vaccines that had been prepared in pre-lyophilization formulations at pH 6.0 or pH 7.2. Controls received identical courses of saline plus adjuvant.

FIG. 14 shows the antibody titres against the Sta011 antigen in mice following immunization with reconstituted lyophilized adjuvanted tetravalent vaccines that had been prepared in pre-lyophilization formulations at pH 6.0 or pH 7.2. Controls received identical courses of saline plus adjuvant.

FIG. 15 shows the antibody titres against the EsxAB antigen in mice following immunization with reconstituted lyophilized adjuvanted tetravalent vaccines that had been prepared in pre-lyophilization formulations at pH 6.0 or pH 7.2. Controls received identical courses of saline plus adjuvant.

FIG. 16 shows the survival rates of immunized mice after S. aureus challenge.

### MODES FOR CARRYING OUT THE INVENTION

Material and Methods

Differential Scanning calorimetry (DSC) analyses were performed with TA Instruments Q2000. 10 μL of the tested solution were loaded in hermetic TO aluminum pans, equilibrated at −80° C. and then heated at different scanning rates (from 10 to 20° C/minute) to 0° C.

Size-Exclusion High Pressure Liquid Chromatography (SE-HPLC) analyses were performed with a Waters Alliance 2695 instrument, by injecting 100 μL of pre-lyophilized formulation. The separation was afforded with the column Phenomenex Biosep SEC-S-3000 in 0.8 mL/minute isocratic conditions (0.1 M sodium phosphate+0.1M sodium chloride, pH 7). Spectra were recorded at 214 nm.

Reversed Phase Chromatography (RP) analyses were performed with a Waters Alliance 2695 instrument, by injecting 50 μL of pre-lyophilized formulation. The separation was afforded with the column ACE 3 C4-300. Flow: 1 mL/minute, in a TFA/acetonitrile gradient.

Capillary Electrophoresis analyses were performed with a Beckman Coulter PA800 instrument, SDS-MW application. The sample was prepared by mixing 90 μL of the pre-lyophilized formulation with 10 μL of TRIS-SDS buffer.

Lyophilization runs were performed in Virtis Genesis EL 25, composed by 5 shelves. 0.3 mL of solution was filled in 2 cc Type I glass vials and siliconed butylic stoppers.

Antigen purity: EsxAB purity=(monomer+dimer)/total proteins.

Antigen selectivity: EsxAB selectivity %=% monomer/% (monomer+dimer).

EsxAB Stability

To investigate the stability of the monomeric and dimeric forms of EsxAB hybrid polypeptide (SEQ ID NO: 250), various stabilizing additives were screened, including sucrose 5/10% w/V, arginine 50/150 mM, EDTA 20 mM,proteases inhibitors mixture, glycerol 10% and citrate 50 mM. All of these were analyzed in 10 mM potassium phosphate buffer at pH6.0.Buffer pH was also tested at pH 5.8 and 5.5. The storage conditions tested were room temperature (RT), 2-8° C. and freeze/thaw cycles.

In all the experiments, partial dimerization and the consequent loss of purity were observed.

Only 20 mM EDTA stabilized the monomer form keeping, as consequence, total purity above 80%.Table 1 shows data obtained from RP-HPLC analyses of experiments containing buffer at pH 6.0 and 0 or 20 mM EDTA carried out at room temperature and at 2-8° C.

TABLE 1
Stability Data on purified EsxAB monomer or dimer at RT and 2-8° C.

| | | Dimer | | Monomer | | Monomer | |
|---|---|---|---|---|---|---|---|
| | | (no | | (no | | (20 mM | |
| | | EDTA) | | EDTA) | | EDTA) | |
| Temperature | Days | Purity % | Selectivity % | Purity % | Selectivity % | Purity % | Selectivity % |
| RT | 0 | 81.9 | 90.2 | 82.6 | 98.0 | 80.0 | 98.0 |
| RT | 1 | 75.9 | 95.8 | n.d. | n.d. | n.d. | n.d. |
| RT | 3 | n.d. | n.d. | 76.5 | 70.8 | 83.0 | 97.1 |
| RT | 7 | 68.5 | 97.4 | 69.8 | 28.6 | 83.5 | 96.3 |
| 2-8° C. | 0 | 81.9 | 90.2 | 82.6 | 98.0 | 80.0 | 98.0 |
| 2-8° C. | 1 | 78.6 | 94.0 | n.d. | n.d. | n.d. | n.d. |
| 2-8° C. | 3 | n.d. | n.d. | 79.8 | 90.5 | 84.0 | 98.0 |
| 2-8° C. | 7 | 75.2 | 96.9 | 77.4 | 54.8 | 84.8 | 97.7 |

To ascertain the minimum concentration of EDTA in the final pre-bulk buffer necessary to keep the product stable, the stability of the monomer EsxAB (stored in 10 mM potassium phosphate at pH6) was studied over a concentration range of 0-20 mM EDTA. Data obtained from RP-HPLC are reported in Table 2.

TABLE 2
Data on stability studies of EsxAB monomers
at different final concentration of EDTA.

| EDTA Final | | Purity | Selectivity |
|---|---|---|---|
| Concentration (mM) | Days | RP-HPLC % | RP-HPLC % |
| 0 | 0 | 81.5 | 97.1 |
| 0 | 3 | 67.0 | 43.4 |
| 0 | 6 | 57.3 | 17.3 |
| 2 | 0 | 82.0 | 97.7 |
| 2 | 3 | 78.5 | 96.2 |
| 2 | 6 | 78.1 | 95.4 |
| 5 | 0 | 81.3 | 97.8 |
| 5 | 3 | 78.1 | 95.9 |
| 5 | 6 | 78.3 | 95.3 |
| 10 | 0 | 81.7 | 97.8 |
| 10 | 3 | 78.3 | 95.9 |
| 10 | 6 | 78.4 | 95.3 |
| 20 | 0 | 81.5 | 97.8 |
| 20 | 3 | 78.9 | 95.9 |
| 20 | 6 | 78.3 | 95.2 |

On the basis of the data obtained, EsxAB monomers can be considered stable in terms of RP-HPLC purity and selectivity at RT for up to 6 days, when the final buffer have an EDTA concentration >2 mM.

Further studies on the stability of the EsxAB monomer in the absence or presence of 5 mM EDTA were carried out. In the presence of EDTA, EsxAB monomer was stable at 2-8° C. for up to 28 days; whereas in the absence of EDTA, there was a significance loss in RP-HPLC selectivity and purity after 7 days. The EsxAB monomer was also stable after up to 5 freeze/thaw cycles; whereas the absence of EDTA led to a slight loss in RP-HPLC selectivity after 3 freeze/thaw cycles.

EsxAB monomer concentrated bulk is therefore stored in 10 mMpotassium phosphate buffer at pH6 and in 5 mM EDTA.

Antigen Stability

The antigens of the compositions used in this experiment are summarized in Table 3 below. All four antigens are recombinant proteins—they were expressed in E.coli and purified from the soluble fraction of total cell extracts.

TABLE 3
Antigens in S. aureus composition

| | | | SEQ |
|---|---|---|---|
| Antigen | Modification in S. aureus | Size/MW | ID NO. |
| HlaH35L | Wild type Hla detoxified by one | 396 amino | 232 |
| | amino acid substation (His35Leu) | acids/33 kDa | |
| EsxAB | Wild type EsxA and EsxB fused | 206 amino | 250 |
| | with a short spacer (ASGGGS) | acids/22.8 kDa | |
| Sta006 | Wild type Sta006 | 288 amino | 248 |
| | | acids/32 kDa | |
| Sta011 | Wild type Sta011 | 234 amino | 249 |
| | | acids/27 kDa | |

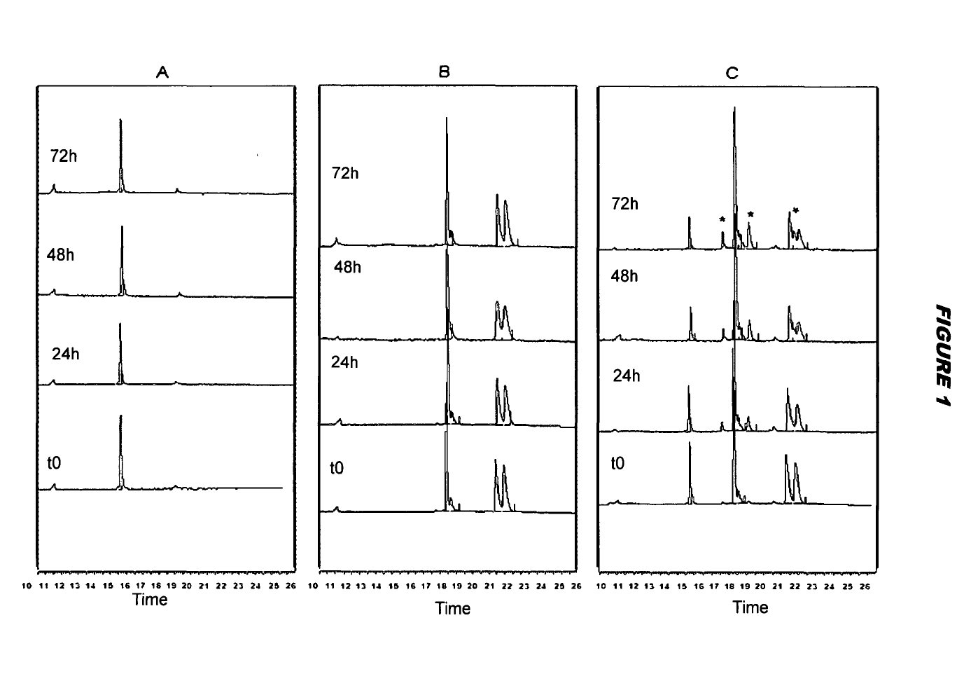

The protein stability of the tetravalent (EsxAB, Sta006, Sta011 and HlaH35L), trivalent (Sta006, Sta011 and HlaH35L) and monovalent (EsxAB) pre-lyophilized formulations in 10 mM potassium phosphate buffer at pH 6 was analysed. FIG. 1 reports the results of capillary electrophoresis analyses. Interestingly, the monovalent and trivalent formulations were stable at 25° C. for up to 72 hours, whereas, in the tetravalent formulation, EsxAB tended to evolve to the dimer and heterodimers of EsxAB with Sta monomers (marked with * in the graphs) appearing over time. The inventors believe that the monomeric EsxAB, together with Sta006 and Sta011, performs redox reactions in the absence of a stabilizing additive.

Impact of EDTA on Formulation

The concentration of EDTA required optimization both to maintain EsxAB in its monomeric form, and to avoid any interference with the other components (i.e. Sta006, Sta011 and HlaH35L) was investigated.

The impact of EDTA on the thermal properties of the formulation was evaluated, by means of Differential Scanning calorimetry (DSC).To spare useful antigenic stock supply, the experiments were carried out using placebo formulations. The placebo formulations include 10 mM potassium phosphate at pH 7.2, 5% sucrose andO, 0.75 mM, 5 mM, 10 mM, 50 mM or 100 mM EDTA, and10 mM potassium phosphate at pH 6.0, 5% sucrose and 5 mM EDTA.

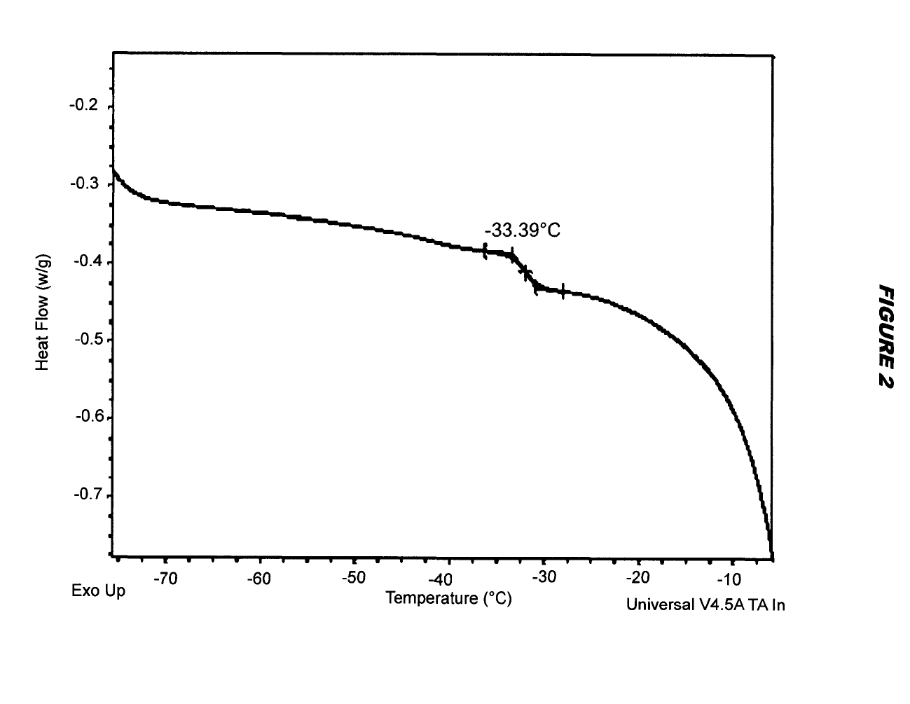

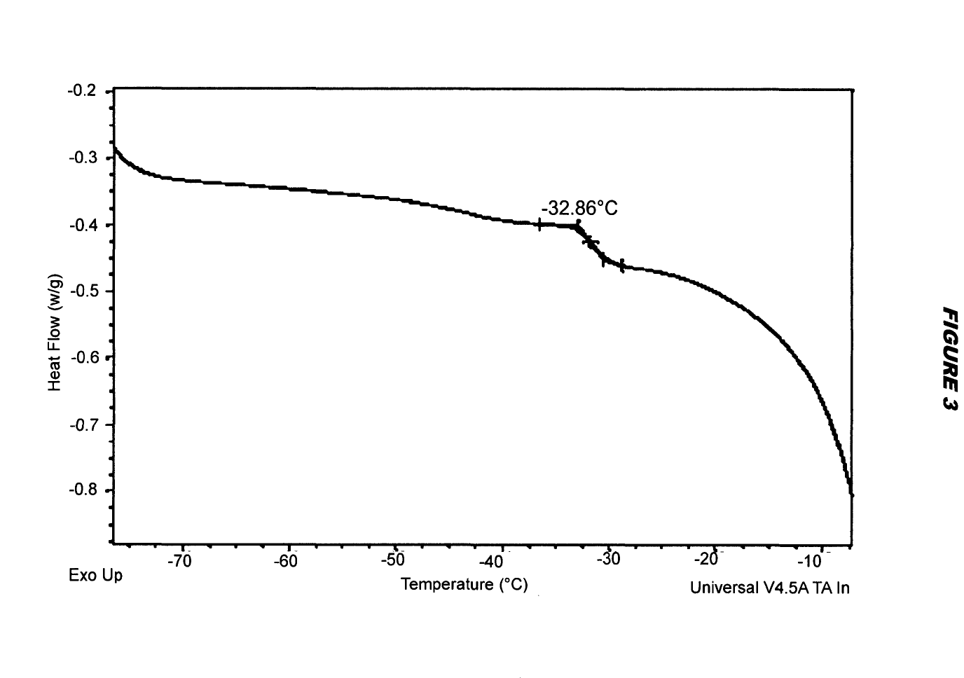

FIGS. 2 and 3 report the data obtained from the heating phase of 0.75 mM and 5 mM EDTA placebo formulations (at pH 7.2), respectively. The onset temperature value represents the glass transition temperature (Tg) of the frozen solution. The Tg values for 0.75 mM (−33.39° C.) and 5 mM (−32.86° C.) EDTA placebo formulations are similar to the Tg value for placebo without EDTA (−33.88° C.).

The results did not show a significant impact for any of the tested EDTA concentrations (at pH 6.0 and pH 7.2) on the thermal behavior of the solution, and there was no undesired plasticizing effect. The sucrose-based lyophilization cycle of the formulation was also not affected by the presence of EDTA.

This conclusion was further confirmed by Freeze-Drying Microscopy (FDM) studies on 5 mM EDTA placebo formulation (at pH 7.2).

The effect of EDTA on the stability of the tetravalent pre-lyophilized formulation was next analyzed. The composition of the tested formulations is listed in Table 4 below.

TABLE 4
Composition of the tested pre-lyophilized formulations (pH 7.2)

| | Concentration | | | | | |
|---|---|---|---|---|---|---|
| | | | 0.75 mM | | 5 mM | |
| Component | Control | | EDTA | | EDTA | |
| Sta006 | 144 | μg/mL | 144 | μg/mL | 144 | μg/mL |
| Sta011 | 144 | μg/mL | 144 | μg/mL | 144 | μg/mL |
| HlaH35L | 144 | μg/mL | 144 | μg/mL | 144 | μg/mL |
| EsxAB | 144 | μg/mL | 144 | μg/mL | 144 | μg/mL |
| EDTA | 0 | | 0.75 | mM | 5 | mM |
| Potassium phosphate | 10 | mM | 10 | mM | 10 | mM |

| | | | |
|---|---|---|---|
| Sucrose | 5% | 5% | 5% |

1) Size Exclusion Chromatography (SEC)-HPLC Analyses

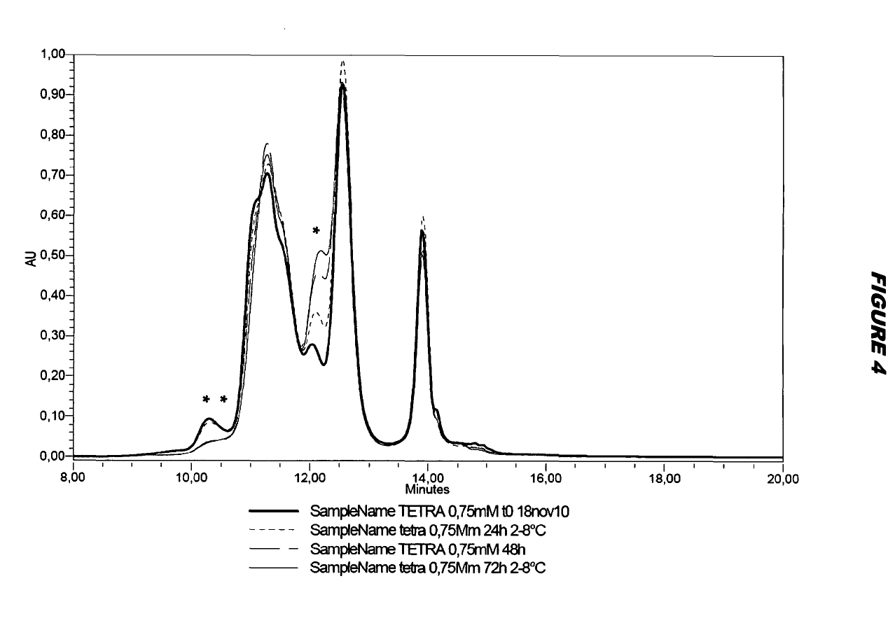

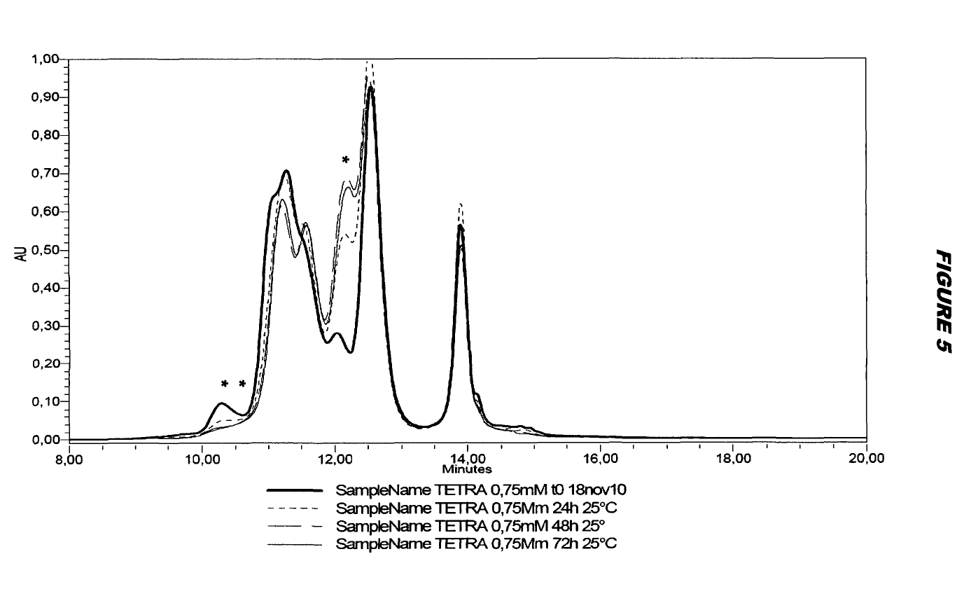

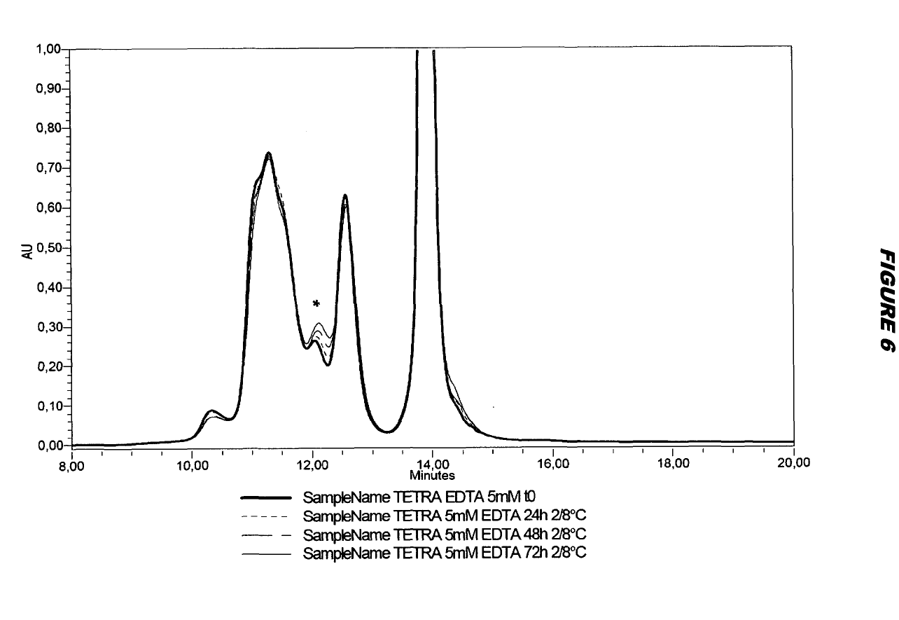

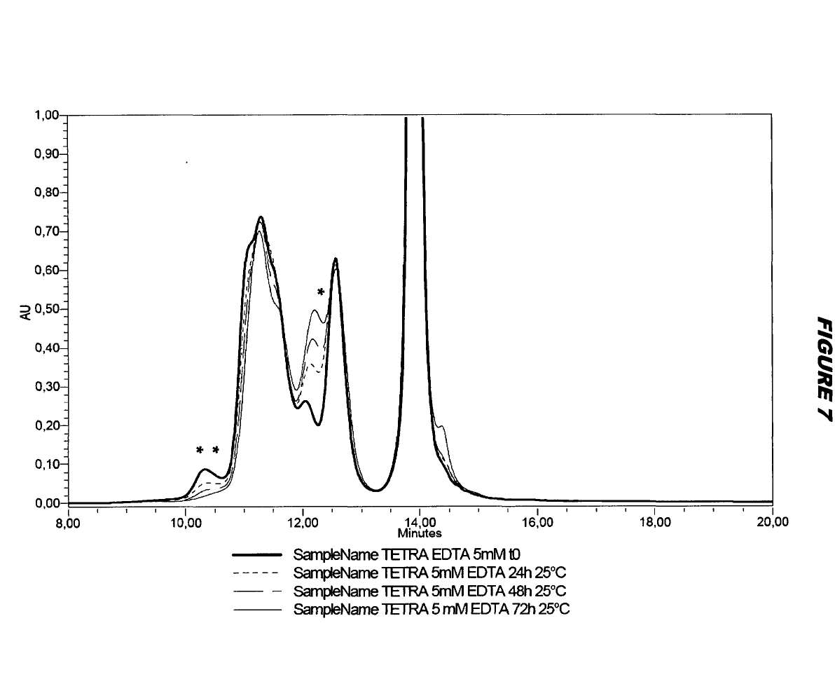

These analyses showed that at t=0 (FIGS. 4-7), the profile of the tetravalent composition is slightly impacted by EDTA: an increment of the area of the first peak (an overlapping of the signals due to Sta006 and Sta011 dimers and partially EsxAB monomer) as well as a decrement of the area of the second peak (HlaH35L and partially EsxAB monomer)were observed.

At 2-8° C. and RT, after 24 h,48 h and 72 h (FIGS. 4-7), a further modification of the peaks profile became evident, showing the presence of a new significant shoulder (marked with * in the graph), at retention times intermediate between the two peaks, probably associated with the formation of monomeric forms of Sta006 andSta011. An aggregation peak (marked with ** in the graph), at low retention time is also evident.

The proteins are more stable at the lower temperature and the higher EDTA concentration. In fact, while the 0.75 mM pre-lyophilized formulations were not stable in the time-span investigated at any temperature, the 5 mM formulation demonstrated good stability but only at 2-8° C. In addition, 10 mM EDTA formulation was analyzed, but the modest increment in the protein stability was considered insufficient to justify the increase in EDTA dosage per injection.

2) Native Gel Analysis

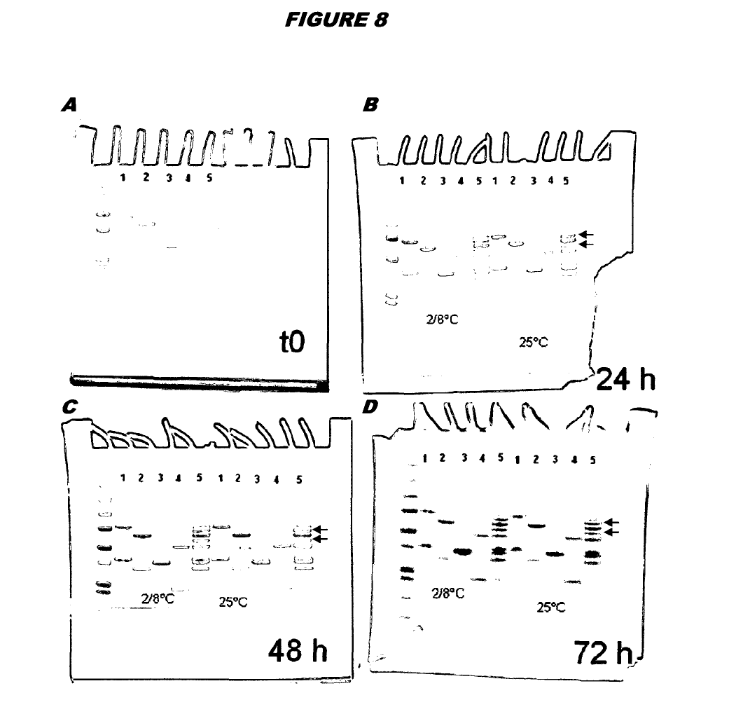

Native gels (no SDS and no reducing agent) of the pre-lyophilized formulations (5 mM EDTA) confirmed the previous data, showing a change in the intensity of the monomer-dimer bands of the proteins Sta006, Sta011 and EsxAB, and the appearance of at least two new bands (see arrows in FIG. 8).

pH Optimization

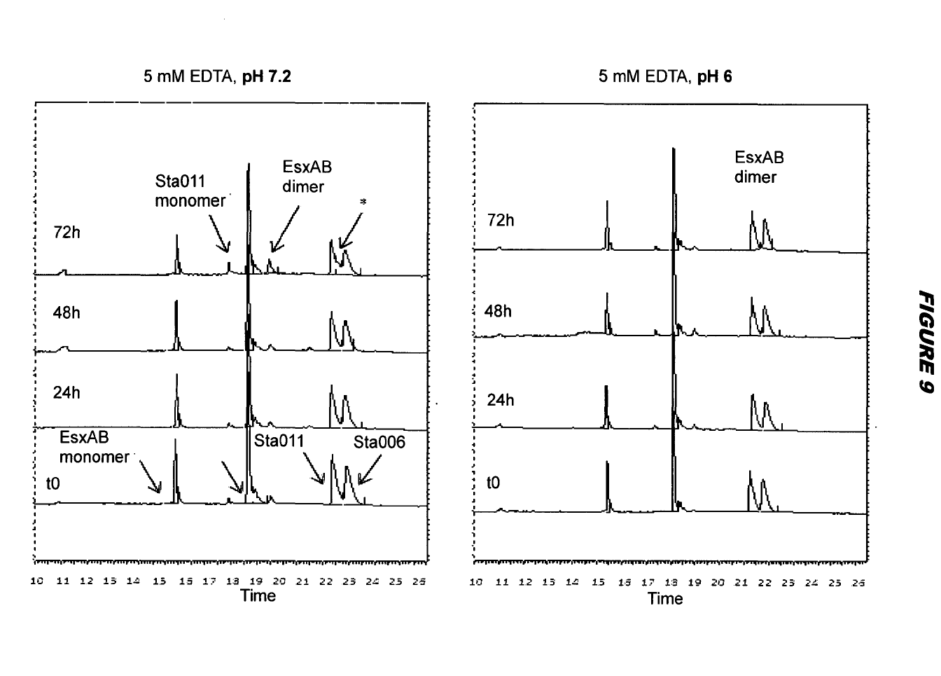

By changing the pH from 7.2 to 6, a further increment of formulation stability is observed, as highlighted in FIG. 9, where profiles recorded with Capillary Electrophoresis of the pre-lyophilized formulations, maintained at RT for up to 72 h, both at pH 7.2 and pH 6, are compared. In particular, at pH 6.0 the increment of the peaks of EsxAB dimer and of Sta011 monomer over time is noticeably lower than at pH 7.2.

The stability of EsxAB at different pH conditions was investigated by measuring protein selectivity, which is a parameter of stability.

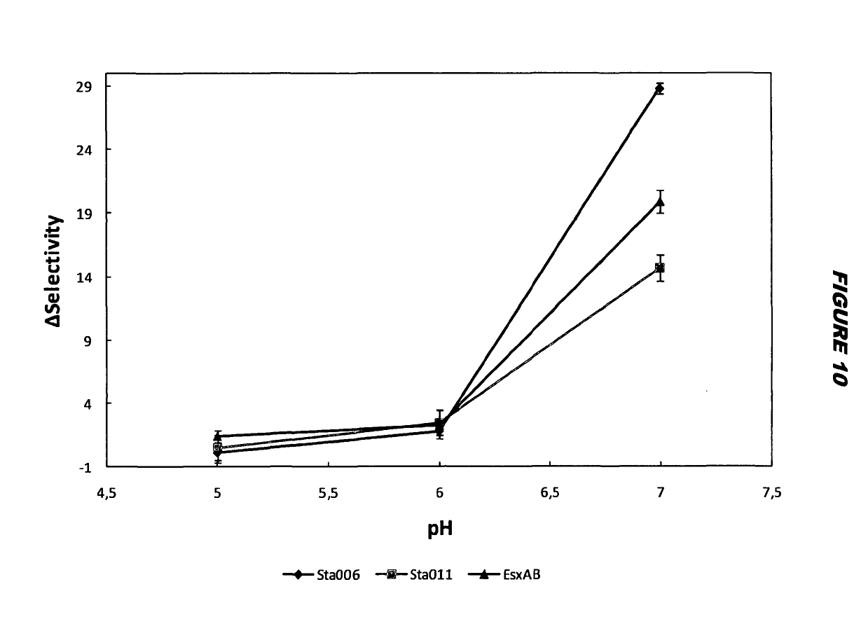

FIG. 10 reports A selectivities (t24 h-t0) for the three proteins: EsxAB, Sta006 and Sta011 (HlaH35L cannot be described in terms of selectivity), recorded by Reversed Phase Chromatography. While the ASelectivity decrement, from pH 7 to 6 is evident (confirmed by p-values=0 for all proteins), the further decrement at pH 5 is at the limit of statistical relevance for two proteins (Sta006: p-value=0.040, Sta011: p-value=0, EsxAB: p-value=0.053).

Given the results of the pH optimization, and since at pH 5 the EDTA-phosphate solution has no buffering capacity, pH 6 was chosen for the final formulation. Moreover, the impact of pH on the thermal properties of this formulation was evaluated by DSC. There was no undesired plasticizing effect, and the sucrose-based lyophilization cycle was not affected.

In summary, the formulation at pH 6 minimizes antigen interactions and guarantees an acceptable stability of the selected pre-lyophilized formulation for up to 72 hours at RT. The composition of the selected pre-lyophilized formulation is summarized in Table 5.

TABLE 5
Final composition of the selected
pre-lyophilized formulation (pH 6)

| Component | Concentration | |
|---|---|---|
| Potassium phosphate | 10 | mM |
| Sta 006 | 144 | μg/mL |
| Sta 011 | 144 | μg/mL |
| Hlah35l | 144 | μg/mL |
| EsxAB | 144 | μg/mL |
| Sucrose | 5% | |
| EDTA | 5 | mM |

TABLE 6
Summary of the parameters for the freeze-drying cycle

| | | | | | Vacuum | |
|---|---|---|---|---|---|---|
| Lyophilization | | | | | (capacitance | |
| step | Temperature | | Time | | manometer) | Rate/Hold |
| Loading | 10° | C. | 1 | min | — | Hold |
| Freezing | −50° | C. | 60 | min | — | Rate |
| | −50° | C. | 60 | min | — | Hold |
| Primary drying | −50° | C. | 15 | min | 75 mT | Hold |
| | −28° | C. | 30 | min | 75 mT | Rate |
| | −28° | C. | 780 | min | 75 mT | Hold |
| Secondary drying | 30° | C. | 180 | min | 75 mT | Rate |
| | 30° | C. | 420 | min | 75 mT | Hold |

| | | |
|---|---|---|
| Total time | ≈26 | h |

The pre-lyophilized formulation is lyophilized according to the freeze-drying cycle in Table 6 to obtain a dried product with cake-like appearance.

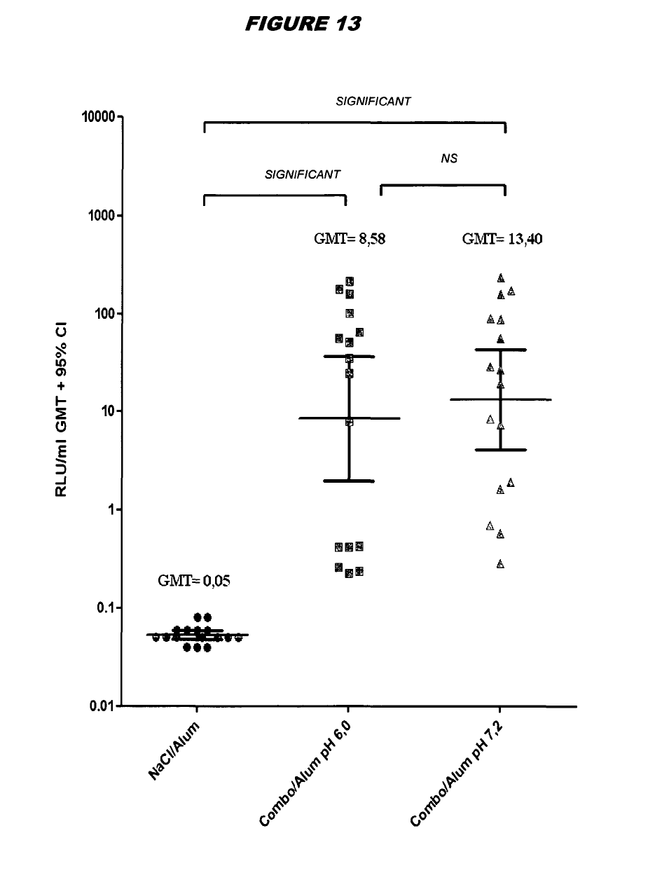

Finally, the protein stability upon lyophilization was verified, by comparing the SEC profiles of the pre-lyophilized formulation (stored at RT for 24 h, to allow the system to reach equilibrium) and of the lyophilized product reconstituted with 0.3 ml- of water. FIG. 13 shows that the chromatograms of the formulations pre-lyophilization (A) and post-lyophilization (B) mirror each other.

Toxicological/Clinical Studies

The amounts of each component per vial (according to the final dosage to be used for toxicological/clinical studies) are listed in Table 7.

TABLE 7
Components of S. aureus lyophilizate.

| Component | Amount/vial | |
|---|---|---|
| Sta006 | 43.2 | μg |
| Sta011 | 43.2 | μg |
| HlaH35L | 43.2 | μg |
| EsxAB | 43.2 | μg |
| Sucrose | 15 | mg |
| EDTA | 0.56 | mg |
| Potassium phosphate | 0.41 | mg |

The lyophilizate is reconstituted with a suitable diluent (e.g. aluminium hydroxide and/or saline solution) for further studies, such as toxicological/clinical studies.

Table 8 reports stability data for the main product quality attributes of the above representative staphylococcus aureus lyophilized vaccine lot stored at 2-8° Cup to 12 months. Table 9 and 10 report data under accelerated conditions (23/27° C. 60±5% relative humidity) up to 6 months and stressed conditions (38/42° C.) up to 4 weeks.

TABLE 8
Stability at 2-8° C. of a S. aureus representative lyophilized lot

| | Measure | Acceptance | Analyzed | | | | | |
|---|---|---|---|---|---|---|---|---|
| Analysis | Unit | criteria | Product | 0 | 3 | 6 | 9 | 12 |
| Residual | (%) | ≦3.0% | Lyophilizate | 0.5 | 1.1 | 1.1 | 1.2 | 1.4 |
| Moisture | | | lot | | | | | |
| RP-HPLC | % | Report result | Sta006 | 92.8 | 90.1 | 92.0 | 92.2 | 91.4 |
| (Selectivity) | | | Sta011 | 95.8 | 95.4 | 96.0 | 96.0 | 95.6 |
| | | | EsxAB | 97.0 | 93.6 | 90.9 | 91.3 | 91.7 |
| | | | HlaH35L | n/a | n/a | n/a | n/a | n/a |
| pH | n/a | 5.5-6.5 | Lyophilizate | 6.1 | 6.1 | 6.1 | 6.1 | 6.1 |
| | | | lot | | | | | |

TABLE 9
Stability at 23/27° C. 60% 60 ± 5% relative humidity of a S. aureus
representative lyophilized lot

| | Measure | Acceptance | Analyzed | | 1 | 3 | 6 |
|---|---|---|---|---|---|---|---|
| Analysis | Unit | criteria | Product | 0 | month | months | months |
| Residual | (%) | ≦3.0% | Lyophilizate | 0.5 | 1.4 | 1.6 | 2.1 |
| Moisture | | | lot | | | | |
| RP-HPLC | % | Report result | Sta006 | 92.8 | 91.0 | 91.5 | 92.2 |
| (Selectivity) | | | Sta011 | 95.8 | 95.7 | 95.6 | 95.9 |
| | | | EsxAB | 97.0 | 94.6 | 91.6 | 89.8 |
| | | | HlaH35L | n/a | n/a | n/a | n/a |
| pH | n/a | 5.5-6.5 | Lyophilizate | 6.1 | 6.1 | 6.1 | 6.1 |
| | | | lot | | | | |

TABLE 10
Stability at 38/42° C. of a S. aureus representative lyophilized lot

| | Meas- | Accept- | | | | |
|---|---|---|---|---|---|---|
| | ure | ance | Analyzed | | 2 | 4 |
| Analysis | Unit | criteria | Product | 0 | weeks | weeks |
| Residual | (%) | ≦3.0% | Lyophilizate | 0.5 | 1.4 | 1.4 |
| Moisture | | | lot | | | |
| RP-HPLC | % | Report | Sta006 | 92.8 | 91.4 | 91.0 |
| (Selectivity) | | result | Sta011 | 95.8 | 95.8 | 95.8 |
| | | | EsxAB | 97.0 | 92.1 | 93.7 |
| | | | HlaH35L | n/a | n/a | n/a |
| pH | n/a | 5.5-6.5 | Lyophilizate | 6.1 | 6.1 | 6.1 |
| | | | lot | | | |

pH monitoring confirms the chemical physical stability of the product and the maintained buffering capacity of phosphate over time under all storage conditions.

Residual moisture shows an increase at 2/8° C. up to 1,4% after 12 months stability time. The increase is more significant under accelerated and stressed conditions. However, all residual moisture values are still below the 3% value accepted by the US Food & Drug Administration and the World Health Organization. Therefore the suitability of the selected lyophilized formulation is established.

Finally, reverse phase chromatography(RP-HPLC) allows the determination of selectivity for the proteins Sta006, Sta011, EsxAB. The above results show that the selectivity is stable under all storage conditions within the time frame investigated. This is a key parameter when monitoring the stability of the whole formulation. Selectivity is expected to slightly decrease over time, in particular at 37° C. The acceptable Δ selectivity value for each protein, with respect to time zero, is ±20%.The data of tables 8, 9, demonstrate the improved stability of staph aureus antigens in presence of EDTA, pH 6 and in combination with a sucrose-based lyophilized formulation with respect to the liquid product. In addition, the data confirm the formulation suitability and its lyoprotector activity upon lyophilization and storage even with residual moisture values close to the upper 3% acceptance criteria.

Short-Term Stability Studies of Lyophilized Vaccine after Reconstitution

The vaccine lyophilizate of Table 7 was reconstituted in a solution of: (1) 2 mg/ml aluminium hydroxide in 7 mg/ml NaCl or (2) saline (9 mg/ml NaCl) to obtain dosages of 36 μg/dose, 12 μg/dose and 4 μg/dose. The characteristics of the reconstituted vaccines were assessed at 0, 3, 8 and 24 hours of storage at two different temperatures 2-8° C. and 36-38° C. The characteristics assessed were pH, osmolality and appearance. For vaccines reconstituted with aluminium hydroxide, percentage of adsorption and particle size distribution were also assessed. For vaccines reconstituted with saline, protein selectivity (by performing reverse phase (RP)-HPLC analysis) and aggregates formation (by performing size-exclusion (SEC)-HPLC analysis) were also assessed.

pH

The pH trend over time provides an indication of the stability of the proteins. The pH value of the reconstituted vaccine was determined according to the internal standard procedure and the European Pharmacopoeia definition. The acceptable pH value for the vaccine is in the range of 5.50≦pH≦6.50.

Osmolality

The osmolality value of the reconstituted vaccine at t=0 was determined according to the internal standard procedure and the European Pharmacopoeia definition.

Appearance

The method is based on the visual inspection of the reconstituted vaccine. At least three containers were examined. Both their colour and transparency were assessed. The contents were examined through the clear colourless walls of the vial, against a black background to assess transparency, and against a white background to reveal coloration, using diffuse daylight. The reconstituted vaccine was defined as “COMPLIANT” when it corresponded to an opalescent liquid with white suspension, free from visible foreign particles.

Percentage of Adsorption on Aluminium Hydroxide

This semi-quantitative method allows the monitoring of the effect of time and temperature on the percentage of proteins adsorbed onto aluminium hydroxide. The analysis was performed using SDS-Page method. It has previously been shown that HlaH35L is not completely adsorbed and remains in the supernatant at a percentage of 10-20% approximately.

Particle Size Distribution

Changes in particle size over time are an indication of instability. The particle size distribution was evaluated using the AccuSizer APS instrument. Before performing each analysis, the reconstituted vaccine suspension was gently shaken for 10 min at room temperature (RT).

Selectivity by RP-HPLC

Reverse phase chromatography allows the determination of selectivity for the proteins Sta006, Sta011, EsxAB. This is a key parameter when monitoring the stability of the whole formulation. Selectivity is expected to slightly decrease over time, in particular at 37° C. The acceptable Δ selectivity value for each protein, with respect to time zero, is ±20%.

Selectivity parameter is determined by using the peak area percentages (area %) of dimer and monomer forms of each protein.HlaH35L antigen exists only in monomeric form, and therefore no selectivity for HlaH35L was determined

Selectivity % (Sta006 and Sta011)=% area dimer/(% area dimer+% area monomer)×100

Selectivity % (EsxAB)=% area monomer/(% area dimer+% area monomer)×100

Size-Exclusion Chromatography

The formation of aggregates during storage was monitored by SEC-HPLC analyses. Chromatograms from all samples were super imposable at up to 3 hours at both temperatures (2-8 and 36-38° C.), while after 8 hours storage at 36-38° C., a slight change of antigen profile was observed.

Results

1) Reconstitution with Aluminium Hydroxide

pH—In all reconstituted samples, at both temperatures (2-8° C. and 36-38° C.) and all time points (0, 3, 8 and 24 hours after reconstitution), pH values remained within the acceptable limits (pH 5.50-6.50). However, there was a trend of increasing pH over time, particularly at 36-38° C.

Osmolality—The osmolality in all samples, measured at time zero, was about 0.2-0.3 osm/kg.

Appearance—All tested samples exhibited a compliant appearance.

Adsorption—A similar trend over time was observed for each temperature. At 2-8° C., the amount of unadsorbed HlaH35L remains stable over time; at 36-38° C., a trend where an increment of non-adsorbed protein of 2.5 folds at 24 h with respect to time zero was observed. However, the amount of antigen adsorbed remains higher than 70%. The adsorption to adjuvant of Sta006, Sta011 and EsxAB antigens was always higher than 90% (i.e. 99-100%).

Particle size distribution—Particle size analysis showed that the mean diameter, for each dosage, was stable over the timespan investigated (up to 24 hours after reconstitution).

Based on the above described results, the inventors concluded that Staphylococcus aureus lyophilized vaccine reconstituted with aluminium hydroxide remained suitable for clinical use up to 24 hours at both tested temperatures (2-8° C. and 36-38° C.).

2) Reconstitution with Saline

pH—In all samples reconstituted with saline solution, pH values remained constant and within the set limits (pH 5.20-6.20) over the time-span investigated (up to 24 hours after reconstitution), upon storage both at 2-8 and 36-38° C.

Osmolality—In all samples, the osmolality, measured at time zero, was about 0.2-0.3 osm/kg.

Appearance—All the samples had a compliant appearance.

RP-HPLC—Reversed phase profiles showed that at 2-8° C., selectivity was constant in the investigated time span, while at 36-38° C., a slight decrement for Sta006 and EsxAB was observed, which became more evident at 24 h. This decrement remained lower than 10% over the investigated timespan.

SEC-HPLC—Chromatograms from all samples were superimposable at up to 3 hours at both temperatures (2-8 and 36-38° C.), while after 8 hours storage at 36-38° C., a slight change of antigen profile was observed.

On the basis of these results, the inventors concluded that Staphylococcus aureus lyophilized vaccine reconstituted with saline solution remained suitable for human use up to 24 h at both tested temperatures (2-8° C. and 36-38° C.).

Immunogenicity Studies in Mice

Immunogenicity and protective efficacy of the tetravalent vaccines with pre-lyophilization formulation at pH 6 and pH 7.2 were investigated.

Both vaccine formulations were lyophilized and reconstituted in a solution of 2 mg/ml aluminium hydroxide in 7 mg/ml NaCl. The pre-lyophilization formulation at pH 6.0 was prepared by mixing 10 mM pH 6.0 potassium phosphate buffer, 100 mM EDTA prepared in 10 mM pH 6.0 potassium phosphate buffer, 35% sucrose prepared in 10 mM pH 6.0 potassium phosphate buffer and 50 μg protein/ml of each antigen (HlaH35L, EsxAB, Sta006 and Sta011). The pre-lyophilization formulation at pH 7.2 was prepared in the same way as above, except that 10 mM pH 7.2 potassium phosphate buffer was used.

CD1 mice were immunized twice via intraperitoneal injection two weeks apart, and each mouse received 200 μl of the formulations. Controls received identical courses of saline plus adjuvant (2 mg/ml aluminium-hydroxide). After the second immunization, antibody titers were determined by Luminex assay in sera of mice bled nine days. Statistical analysis was performed by Mann-Whitney U test.

Immunized animals were challenged on day 24 by intraperitoneal injection of a bacterial suspension of S. aureus. Cultures of S. aureus were centrifuged, washed twice and diluted in PBS before challenge. Mice were infected with approximately 2 to 5*10⁸ CFU of S.aureus. Survival rates were analyzed by Fisher's exact test. Mice were daily monitored and euthanized according to humane endpoints, in agreement with Novartis Animal Welfare Policies.

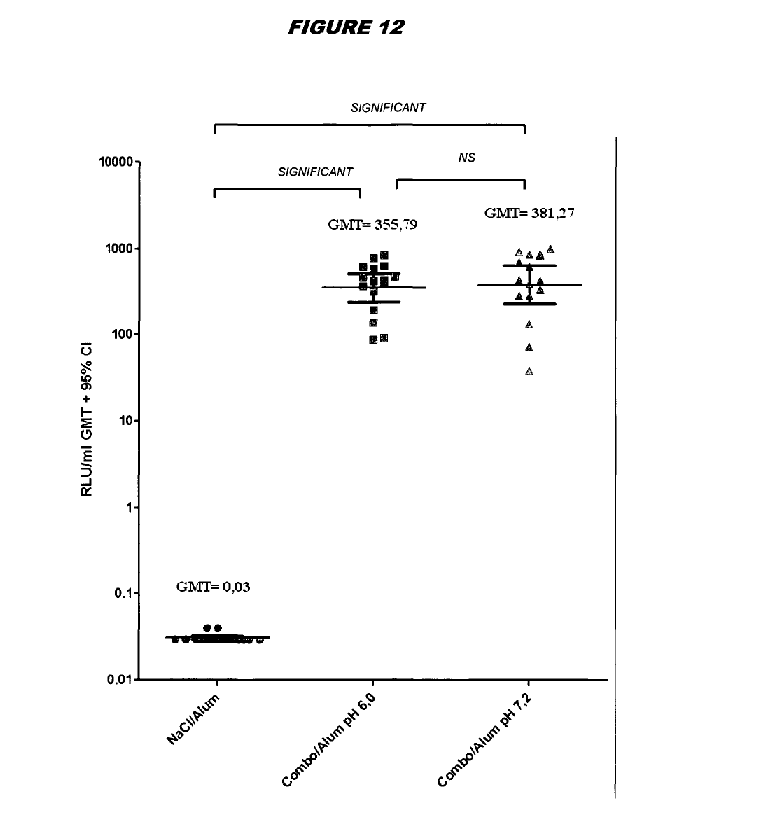

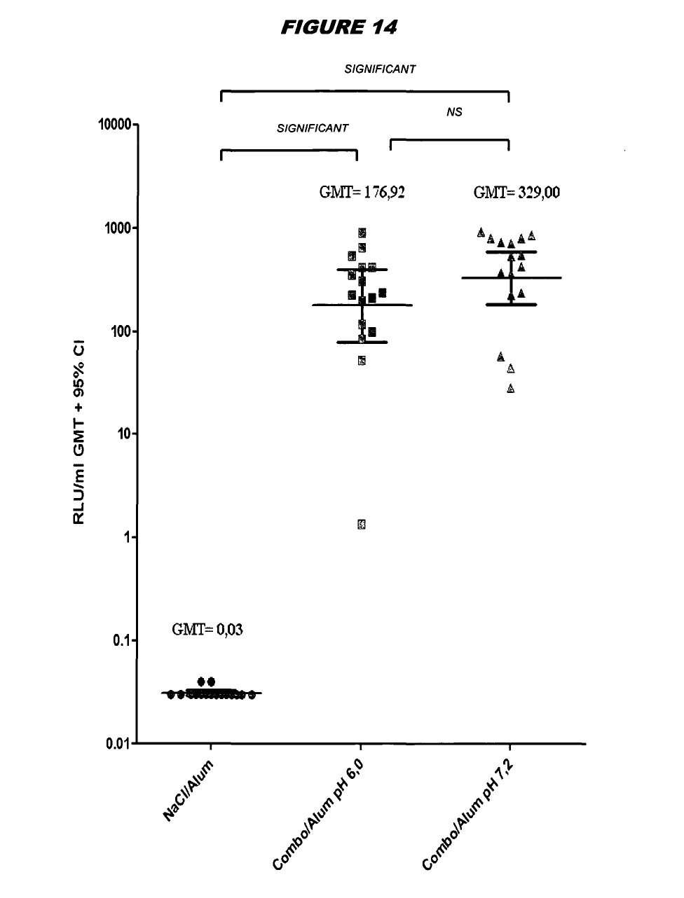

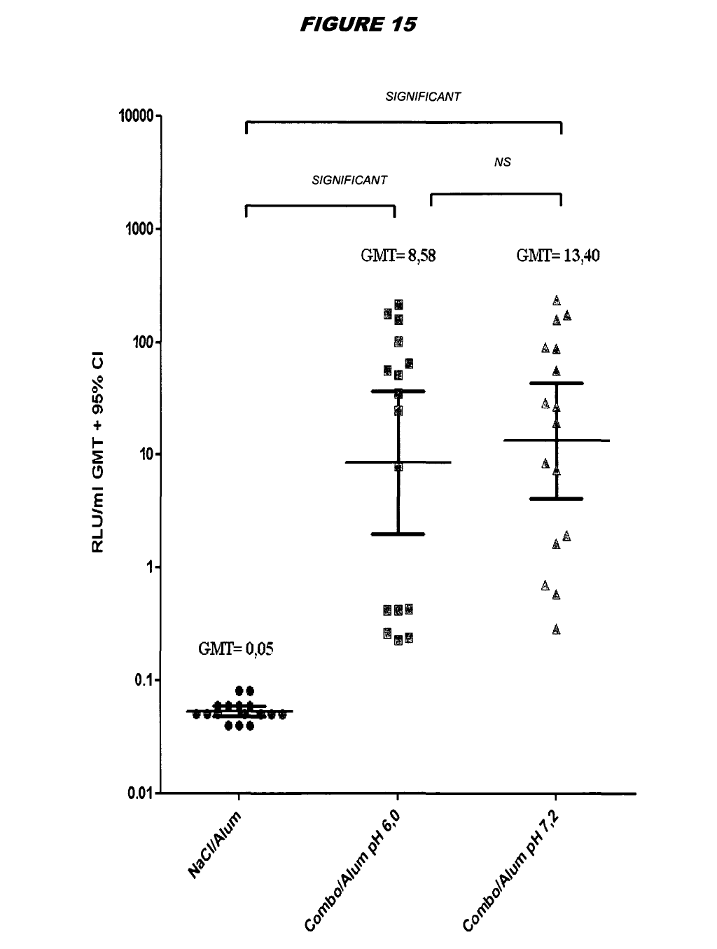

FIGS. 12-15 report antibody titres of mice following immunization. The antibody titres for each antigen were not different significantly between the two vaccines which had been prepared in pre-lyophilization formulations at pH 6.0 and pH 7.2.

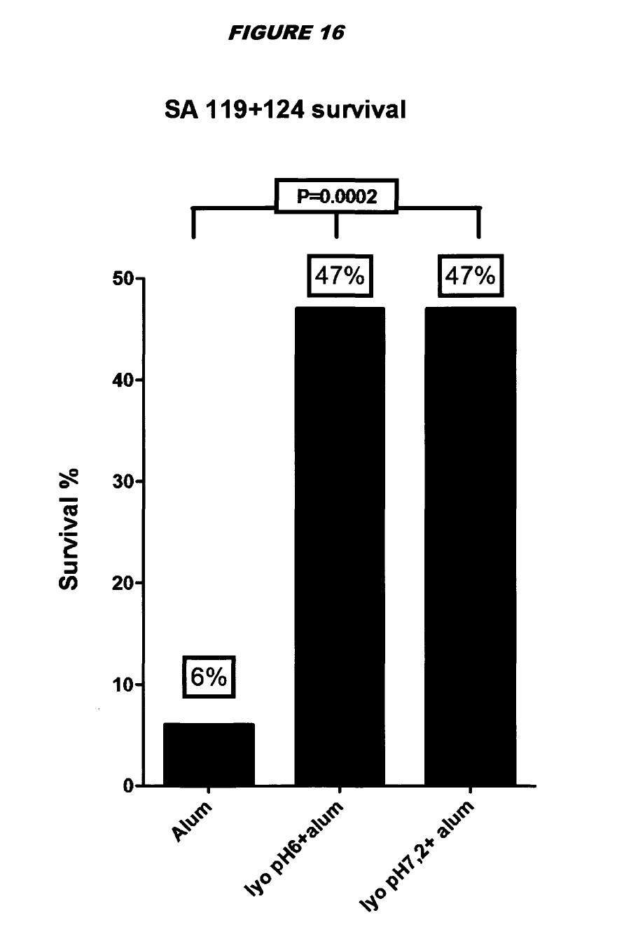

FIG. 16 reports the survival rates of immunized mice after S. aureus challenge. The protective efficacy was not significantly different between vaccines which had been prepared in pre-lyophilization formations at pH 6.0 and pH 7.2.

Therefore changing the pre-lyophilization buffer from pH 7.2 to pH 6.0 did not significantly alter the protective efficacy of the immunization composition.

It will be understood that the invention is described above by way of example only and modifications may be made whilst remaining within the scope and spirit of the invention.

[1] Harroet al. (2010) Clin Vaccine Immunol 17:1868-74.

[2] Kuklinet al. (2006) Infect Immun. 74(4):2215-23.

[3] Sheridan (2009) Nature Biotechnology 27:499-501.

[4] WO2010/119343.

[5] Sebulsky& Heinrichs (2001) J Bacterioll 83:4994-5000.

[6] Sebulskyet al. (2003) J BiolChem278:49890-900.

[7] Rable&Wardenburg (2009) Infect Immun 77:2712-8.

[8] WO2007/145689.

[9] WO2009/029831.

[10] Kurodaet al. (2001) Lancet 357:1225-1240.

[11] Needleman&Wunsch (1970) J. Mol. Biol. 48, 443-453.

[12] Rice et al. (2000) Trends Genet 16:276-277.

[13] WO2008/019162.

[14] U.S. Pat. No. 6,355,271.

[15] WO00/23105.

[16] Vaccine Design . . . (1995) eds. Powell & Newman.ISBN: 030644867X. Plenum.

[17] WO2011/027222.

[18] WO90/14837.

[19] WO90/14837.

[20] Podda& Del Giudice (2003) Expert Rev Vaccines 2:197-203.

[21] Podda (2001) Vaccine 19: 2673-2680.

[22] Vaccine Design: The Subunit and Adjuvant Approach (eds. Powell & Newman) Plenum Press 1995 (ISBN 0-306-44867-X).

[23] Vaccine Adjuvants: Preparation Methods and Research Protocols (Volume 42 of Methods in Molecular Medicine series). ISBN: 1-59259-083-7. Ed. O′Hagan.

[24] WO2008/043774.

[25] Allison &Byars (1992) Res Immunol 143:519-25.

[26] Hariharanet al. (1995) Cancer Res 55:3486-9.

[27] US-2007/014805.

[28] US-2007/0191314.

[29] Suliet al. (2004) Vaccine 22(25-26):3464-9.

[30] WO95/11700.

[31] U.S. Pat. No. 6,080,725.

[32] WO2005/097181.

[33] WO2006/113373.

[34] Han et al. (2005) Impact of Vitamin E on Immune Function and Infectious Diseases in the Aged at Nutrition, Immune functions and Health EuroConference, Paris, 9-10 June 2005.

[35] U.S. Pat. No. 6,630,161.

[36] WO2006/110603.

[37] Stranger-Jones et al. (2006) PNAS USA 103:16942-7.

[38] Wardenburget al. (2007) Infect Immun 75:1040-4.

[39] Gennaro (2000) Remington: The Science and Practice of Pharmacy. 20th edition, ISBN: 0683306472.

[40] Methods In Enzymology (S. Colowick and N. Kaplan, eds., Academic Press, Inc.)

[41] Handbook of Experimental Immunology, Vols. I-IV (D. M. Weir and C. C. Blackwell, eds, 1986, Blackwell Scientific Publications)

[42] Sambrooket al. (2001) Molecular Cloning: A Laboratory Manual, 3rd edition (Cold Spring Harbor Laboratory Press).

[43] Handbook of Surface and Colloidal Chemistry (Birdi, K. S. ed., CRC Press, 1997)

[44] Ausubelet al. (eds) (2002) Short protocols in molecular biology,5th edition (Current Protocols).

[45] Molecular Biology Techniques: An Intensive Laboratory Course, (Ream et al., eds., 1998, Academic Press)

[46] PCR (Introduction to Biotechniques Series), 2nd ed. (Newton & Graham eds., 1997, Springer Verlag)

[47] Geysenet a/. (1984) PNAS USA 81:3998-4002.

[48] Carter (1994) Methods Mol Bio136:207-23.

[49] Jameson, B A et al. 1988, CABIOS 4(1):181-186.

[50] Raddrizzani& Hammer (2000) Brief Bioinform 1(2):179-89.

[51] Bublilet al. (2007) Proteins 68(1):294-304.

[52] De Lallaet al. (1999) J. Immunol. 163:1725-29.

[53] Kwok et al. (2001) Trends Immunol22:583-88.

[54] Brusicet al. (1998) Bioinformaticsl4(2):121-30

[55] Meisteret al. (1995) Vaccine 13(6):581-91.

[56] Roberts et al. (1996) AIDS Res Hum Retroviruses 12(7):593-610.

[57] Maksyutov&Zagrebelnaya (1993) Comput ApplBiosci9(3):291-7.

[58] Feller& de la Cruz (1991) Nature 349(6311):720-1.

[59] Hopp (1993) Peptide Research 6:183-190.

[60] Welling et al. (1985) FEBS Lett. 188:215-218.

[61] Davenport et al. (1995) Immunogenetics 42:392-297.

[62] Tsurui & Takahashi (2007) J Pharmacol Sci. 105(4):299-316.

[63] Tong et al. (2007) Brief Bioinform. 8(2):96-108.

[64] Schirle et al. (2001) J Immunol Methods. 257(1-2):1-16.

[65] Chen et al. (2007) Amino Acids 33(3):423-8.

[66] Current Protocols in Molecular Biology (F. M. Ausubelet al., eds., 1987) Supplement 30

[67] Smith & Waterman (1981) Adv. Appl. Math.2: 482-489.

1-15. (canceled)

16. A composition comprising an EsxA antigen, an EsxB antigen and a stabilizing additive, wherein the composition is either in:
(a) an aqueous form, wherein the pH of the composition is between 5 and 6.5; or,
(b) a lyophilized form.

17. The composition of claim 16, further comprising a Sta006 antigen, a Sta011 antigen, a Hla antigen, or any combination thereof.

18. The composition of claim 17, wherein the Sta006 forms a homodimer.

19. The composition of claim 17, wherein the Sta006 and the Sta011 forms a heterodimer.

20. The composition of claim 16, wherein the EsxA antigen and the EsxB antigen are linked to form a hybrid polypeptide.

21. The composition of claim 20, wherein the hybrid polypeptide forms a homodimer.

22. The composition of claim 20, wherein the composition comprises a fusion protein comprising the hybrid polypeptide.

23. The composition of claim 22, wherein the fusion protein further comprises a tag.

24. The composition of claim 16, wherein the stabilizing additive is EDTA.

25. The composition of claim 16, further comprising an adjuvant, a saccharide, or combination thereof.

26. The composition of claim 25, wherein the adjuvant is an aluminum salt adjuvant or an oil-in-water adjuvant.

27. A method for eliciting an immune response in a mammal, the method comprising a step of:
administering to a mammal one or more doses of an immunogenic composition comprising an EsxA antigen, an EsxB antigen and a stabilizing additive, in an amount effective to treat or prevent an S. aureus infection in the mammal.

28. The method of claim 27, wherein the immune response comprises a systemic immune response, a mucosal immune response, or combination thereof.

29. The method of claim 27, wherein the immune response comprises a TH1 immune response, TH2 immune response, or combination thereof.

30. The method of claim 28, wherein the mucosal immune response comprises a TH2 immune response.

31. The method of claim 28, wherein the mucosal immune response comprises an increased production of IgA.

32. The method of claim 27, wherein the S. aureus infection comprises a skin infection, pneumonia, meningitis, osteomyelitis endocarditis, toxic shock syndrome, septicaemia, or any combinations thereof.
